(19) United States
(12) Patent Application Publication
(10) Pub. No.: US 2006/0019907 A1
(43) Pub. Date: Jan. 26, 2006

(54) Guggulsterone: an inhibitor of nuclear factor - kappaB and IkappaBalpha kinase activation and uses thereof

(75) Inventors: Bharat B. Aggarwal, Houston, TX (US); Shishir Shishodia, Houston, TX (US)

Correspondence Address: Fulbright & Jaworski L.L.P.;Fulbright Tower, Suite 5100, 1301 McKinney, Houston, TX, 77010-3095, US

(73) Assignee: Research Development Foundation, Houston, TX (US)

(21) Appl. No.: 11/179,321

(22) Filed: Jul. 12, 2005

Related U.S. Application Data
(60) Provisional application No. 60/587,187, filed on Jul. 12, 2004.

(51) Int. Cl. A61K 31/56; A61K 31/704

(52) U.S. Cl. 514/26; 514/169

(57) ABSTRACT

The present invention provides an inhibitor of NF-κB, guggulsterone and its analogs. Guggulsterone suppresses NF-κB activation induced by TNF, phorbol ester, okadaic acid, cigarette smoke, H₂O₂ and IL-1β, as well as constitutive NF-κB activation expressed in most tumor cells. One mechanism by which guggulsterone inhibits activation of NF-κB is through suppression of IκBα phosphorylation and IκBα degradation. NF-κB-dependent gene transcription is modulated by guggulsterone and its analogs. In particular, induction by TNF, TNFR1, TRADD, TRAF2, NIK and IKK, is modulated by guggulsterone and its analogs. In addition, guggulsterone decreased the expression of genes involved in anti-apoptosis (IAP1, XIAP, Bfl-1/A1, bcl-2, cFLIP, survivin), proliferation (cyclin D1, c-myc) and metastasis (MMP-9, COX2 and VEGF).

This application claims priority to U.S. Provisional Patent Application Ser. No. 60/587,187, filed Jul. 12, 2004, which is incorporated herein by reference in its entirety.

This invention was produced in part using funds obtained through a grant (CA91844) from the National Institutes of Health. Consequently, the federal government has certain rights in this invention.

### BACKGROUND OF THE INVENTION

I. Field of the Invention

The present invention relates generally to the molecular biology of nuclear factor-kappa B (NF-κB). More specifically, the present invention relates to guggulsterone, a compound that can selectively inhibit NF-κB and IκBα kinase activation, downregulate NF-κB mediated gene expression and enhance apoptosis induced by carcinogens and inflammatory agents.

II. Description of the Related Art

Of the 121 prescription drugs in use today for cancer treatment, 90 are derived from plant species. Almost 74% of these were discovered by investigating a folklore claim (Craig, 1997 and 1999). Paclitaxel is perhaps the most recent example. Between 1981 and 2002, 48 of 65 of all drugs approved for the therapy of cancer were natural products, based on natural products, or mimicked natural products in one form or another (Newman et al., 2003). The mechanism of action and the molecular targets of these phytochemicals is under constant investigation. One such phytochemical is guggulsterone.

Guggulsterone (4,17(20)-pregnadiene-3,16-dione) and isomers thereof are a plant sterol derived from the gum resin (guggulu) of the tree Commiphora mukul. The resin of the C. mukul tree has been used in Ayurvedic medicine for centuries to treat a variety of ailments including obesity, bone fractures, arthritis, inflammation, cardiovascular disease, and lipid disorders (Urizar et al., 2003; Sinal et al., 2002). Gujral et al. have demonstrated the anti-arthritic and anti-inflammatory activity of gum guggulu (Gurjal et al., 1960). Sharma et al. have shown its activity in experimental arthritis induced by mycobacterial adjuvant (Sharma et al., 1977). The effectiveness of guggulu for treating osteoarthritis of the knee has also been demonstrated (Singh et al., 2003). Recent studies have shown that guggulsterone is an antagonist for bile acid receptor farnesoid X receptor (FXR) (Urizar et al., 2002; Wu et al., 2002). Other studies have shown that guggulsterone enhances transcription of the bile salt export pump (Cui et al., 2003), and is thus, an important regulator of cholesterol homeostasis. Furthermore, Meselhy et al. have shown that guggulsterone can suppress inflammation by inhibiting inducible nitric oxide synthetase (iNOS) expression induced by LPS in macrophages (Meselhy et al., 2003). A number of inflammatory diseases are mediated through the activation of NF-κB, a nuclear transcription factor (Yamamoto, 2001; Aggarwal et al., 2004).

In cells at rest, NF-κB is present in the cytoplasm, when activated NF-κB is translocated to the nucleus where signal transduction may be propagated (Yamamoto, 2001; Aggarwal et al., 2004). Currently NF-κB consist of a family of Rel-domain containing proteins, Rel A (also called p65), Rel B, c-Rel, p50 (also called NF-κB1), and p52 (also called NF-κB2). Similarly, a family of anchorin-domain-containing proteins has been identified, which keep NF-κB in its inactive state within the nucleus. These include IκBα, IκBβ, IκBγ, IκBαε, bcl-3, p105 and p100. Most carcinogens, inflammatory agents, and tumor promoters including cigarette smoke, phorbol ester, okadaic acid, H₂O₂, and TNF have been shown to activate NF-κB. Under resting conditions, NF-κB consists of a heterotrimer of p50, p65 and IκBα in the cytoplasm. The phosphorylation, ubiquitination, and degradation of IκBα in conjunction with the phosphorylation of p65 lead to the translocation of NF-κB to the nucleus where it binds to specific response elements in the DNA. The phosphorylation of IκBα is catalyzed by IKK which consists of three subunits IKKα, IKKβ and IKKγ. Gene deletion studies have shown that IKKβ is essential for NF-κB activation by most agents. The kinase that induces the phosphorylation of p65 is controversial, but IKKβ, protein kinase C, and protein kinase A have been implicated (Karin et al., 2002; Garg et al., 2002). NF-κB has been shown to regulate the expression of a number of genes whose products are involved in tumorigenesis (Yamamoto, 2001; Aggarwal et al., 2004; Karin et al., 2002; Garg et al., 2002). These include anti-apoptotic genes (e.g., ciap, survivin, traf bcl-2 and bcl-x1); COX2; MMP-9; as well as genes encoding adhesion molecules, chemokines, inflammatory cytokines and cell cycle regulatory genes (e.g., cyclin D1).

Because NF-κB has been implicated in obesity (Yuan et al., 2001), hyperlipidemia (Thurberg et al., 1998), atherosclerosis (Brand et al., 1996), osteoarthritis (Miagkov et al., 1998) and bone loss (Abu-Amer et al., 1997), it is proposed that guggulsterone may mediate its effects through the suppression of NF-κB activation. However, the prior art is deficient in describing the effect of guggulsterone on NF-κB activation, particularly activation induced by inflammatory agents and carcinogens.

### SUMMARY OF THE INVENTION

The present invention fulfills a need and desire in the art for modulating NF-κB activity. As described herein, guggulsterone inhibits activation of NF-κB through suppression of IκBα kinase, IκBα phosphorylation, IκBα degradation, and p65 nuclear translocation. Guggulsterone also abrogates the expression of NF-κB-regulated gene products that inhibit apoptosis, promote inflammation, and promote tumor metastasis. Guggulsterone has been recently shown to antagonize the farnesoid X receptor (FXR) and decrease the expression of bile acid-activated genes (Urizar et al., 2002; Cui et al., 2003). Because activation of NF-κB has been closely linked with inflammatory diseases affected by guggulsterone, it is proposed that it may modulate NF-κB activation. In one embodiment of the invention, guggulsterone is use to modulate the activation of NF-κB and NF-κB activity induced by inflammatory agents and carcinogens.

Electrophoretic mobility gel shift assays show that guggulsterone suppresses NF-κB activation induced by TNF, phorbol ester, okadaic acid, cigarette smoke, hydrogen peroxide and interleukin 1β. The affects of guggulsterone on NF-κB activation are not cell type-specific as both epithelial and leukemia cells show inhibition of NF-κB activity. In certain aspects, guggulsterone may be used to suppress constitutive NF-κB activation in tumor cells. In a further aspect, guggulsterone or a derivative thereof may be administered as composition having a concentration of 1 μM-5 mM; 5 μM-1 mM; 10 μM-500 μM, 25 μM-250 μM; 40 μM-100 μM; or 50 μM guggulsterone in a volume of 0.5, 1, 1.5, 2, 2.5, 3, 3.5, 4, 5, 10, 20 milliliters or more. In certain aspects of the invention a patient or a subject is administered a guggulsterone or guggulsterone analog at a dose of 0.01, 0.1, 0.5, 1, 5, 10, 25, 50, 100 mg/kg to 100, 200, 300, 400, 500, 750, or 1000 mg/kg, including any dose there between. A cell, organ, or patient may be exposed or treated for approximately 1, 2, 3, 4, 5, 10, 12, 24, 48 or more hours. In a particular embodiment, activation of NF-κB acts through suppression of IκBα phosphorylation, degradation of non-phosphorylated IκBα, or both IκBα phosphorylation and degradation of non-phosphorylated IκBα. In certain cases, guggulsterone completely suppresses the TNF-induced Akt kinase and IκBα kinase activation, thus suppressing p65 phosphorylation and nuclear translocation. NF-κB-dependent gene transcription induced by TNF, TNFR1, TRADD, TRAF2, NIK and IKK, may also be modulated, decreased, attenuated, or blocked by guggulsterone, without affecting p65-mediated gene transcription. The activity of COX2 promoter, which has NF-κB-binding site, may also be suppressed leading to suppression of COX2 expression. In addition, guggulsterone may be used to decrease the gene expression of anti-apoptotic genes (IAP1, XIAP, Bfl-1/A1, bcl-2, cFLIP, survivin), proliferative genes (cyclin D1, c-myc) and metastatic genes (MMP-9, COX2 and VEGF); which correlates with enhancement of apoptosis induced by TNF and anticancer agents, such as chemotherapeutic, radiotherapeutic, and other anti-cancer agents. In still a further aspect, it is contemplated that treatment with guggulsterone or its analogs inhibits RANKL-induced osteoclastogenesis.

A further embodiment of the invention, includes methods of inhibiting NF-κB activation or inhibiting the inactivation of inhibitors of NF-κB in a cell, by contacting the cell with guggulsterone or a guggulsterone analog. In certain aspects of the invention, NF-κB activation includes activation induced by carcinogens such as phorbol ester, okadaic acid, and cigarette smoke, as well as activation induced by inflammatory stimuli, such as TNF, IL-1β and H₂O₂. In other aspects, NF-κB activity is the constitutive NF-κB activity present in some cells, for example, constitutive NF-κB activity that occurs in most tumor cells. In still further aspects, guggulsterone or a guggulsterone analog is contemplated as an agent to modulate, suppress, or down regulate NF-κB-dependent gene transcription. Particularly expression (transcription) induced by TNF, TNFR1, TRADD, TRAF2, NIK, and IKK, expression of anti-apoptotic genes (IAP1, XIAP, Bfl-1/A1, bcl-2, cFLIP, survivin), expression of proliferative genes (cyclin D1, c-myc) and expression of metastatic genes (MMP-9, COX2 and VEGF).

In another embodiment of the present invention, there is provided a method of increasing the effect of an apoptosis-inducing agent in a cell, by contacting the cell with TNF, therapeutic agent, or a chemotherapeutic agent (paclitaxel or doxorubicin) in combination with guggulsterone or a guggulsterone analog.

In yet another embodiment of the present invention, guggulsterone or a guggulsterone analog can be used to treat cancer in an individual. It is well known in the art that NF-κB activation plays an important role in cancer development, and inhibition of NF-κB activities is generally believed to be beneficial in cancer treatment. Moreover, guggulsterone or a guggulsterone analog can be used in combination with various known anti-cancer treatments, such as chemotherapy, to enhance the activity of a cancer treatment.

In still yet another embodiment of the present invention, there is provided a method of inhibiting osteoclastogenesis through activation of NF-κB in a cell, by contacting the cell with guggulsterone or a guggulsterone analog.

Embodiments of the invention also include administering guggulsterone to a cell, in particular a multiple myeloma cell, and modulating or inhibiting the activation of STAT-3. In certain aspects the administration of guggulsterone or analogs thereof may be used in treating various cancers with associated STAT-3 activation, in particular multiple myeloma.

Other and further aspects, features, and advantages of the present invention will be apparent from the following description of the presently preferred embodiment of the invention.

Abbreviations used herein are as follows: NF-κB, nuclear factor-kappa B; IκB, inhibitory subunit of NF-κB; SEAP, secretory alkaline phosphatase; IKK, IκBα kinase; COX-2, cyclooxygenase-2; MMP-9, matrix metalloproteinase-9; TNF, tumor necrosis factor; TNFR, TNF receptor; TRADD, TNFR-associated death domain; TRAF2, TNFR-associated factor; NIK, NF-κB-inducing kinase; PMA, phorbol myristate acetate; FBS, fetal bovine serum; SDS, sodium dodecyl sulfate; BSA, bovine serum albumin; PMA, phorbol myristate acetate; EMSA, electrophoretic mobility shift assay.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIGS. 1A-1B. FIG. 1A shows structure of guggulsterone. FIG. 1B shows guggulsterone blocked (inhibited) NF-κB activation induced by PMA, IL-1β, okadaic acid, and H₂O₂. H1299 cells (2×10⁶/ml) were pre-incubated for 4 h at 37° C. with 50 μM guggulsterone and then treated with PMA (100 ng/ml, 1 h), interleukin 1β (100 ng/ml, 1 h), okadaic acid (500 nM, 4 h), and H₂O₂ (250 μM, 1 h). Nuclear extracts were prepared and tested for NF-κB activation.

FIGS. 2A-2B. FIG. 2A shows guggulsterone-mediated suppression of inducible NF-κB activation is not cell type specific. Two million A549, Jurkat or KBM-5 cells were pre-treated with 50 μM guggulsterone for 4 hours and then treated with 0.1 nM TNF for 30 min. The nuclear extracts were then prepared and assayed for NF-κB by EMSA. FIG. 2B shows guggulsterone can also suppress constitutively active NF-κB in tumor cells. Two million multiple myeloma and head and neck squamous cell carcinoma cells were exposed to 50 μM guggulsterone for 4 hours and then nuclear extracts were prepared and assayed for NF-κB by EMSA.

FIGS. 3A-3D. FIG. 3A shows guggulsterone inhibited TNF-dependent NF-κB activation in a dose-dependent manner. H1299 cells (2×10⁶/ml) were pre-incubated with different concentrations of guggulsterone for 4 hours at 37° C. and then treated with 0.1 nM TNF for 30 min. Nuclear extracts were prepared and tested for NF-κB activation. FIG. 3B shows guggulsterone inhibited TNF-dependent NF-κB activation in a time-dependent manner. H1299 cells (2×10⁶/ml) were pre-incubated with 50 μM guggulsterone for the indicated times at 37° C. and then treated with 0.1 nM TNF for 30 min at 37° C. Nuclear extracts were prepared and then tested for NF-κB activation. FIG. 3C shows TNF-induced NF-κB consists of p50 and p65. Nuclear extracts from H1299 cells (2×10⁶/ml) treated or not treated with 0.1 nM TNF for 30 min were incubated with the antibodies indicated for 30 min at room temperature, and the complex was analyzed by supershift assay. FIG. 3D shows guggulsterone does not modulate the ability of NF-κB to bind to the DNA. Nuclear extracts from H1299 cells (2×10⁶/ml) treated or not treated with 0.1 nM TNF for 30 min were treated with the indicated concentrations of guggulsterone for 4 hours at room temperature and then assayed for DNA binding by EMSA.

FIGS. 4A-4C. FIG. 4A shows guggulsterone inhibited TNF-induced phosphorylation and degradation of IκBα. H1299 cells (2×10⁶/ml) were incubated with 50 μM guggulsterone for 4 hours at 37° C., treated with 0.1 nM TNF for different times as indicated at 37° C., and then tested for IκBα (upper panel) and phosphorylated IκBα (middle panel) in cytosolic fractions by Western blot analysis. Equal protein loading was evaluated by β-actin (lower panel). FIG. 4B shows guggulsterone inhibited TNF-induced IκBα kinase activity. H1299 cells (2×10⁶/ml) were treated with 50 μM guggulsterone for 4 h and then treated with 0.1 nM TNF for different time intervals. Whole-cell extracts were prepared and 200 μg of extract was immunoprecipitated with antibodies against IKKα and IKKβ. Thereafter immune complex kinase assay was performed. To examine the effect of guggulsterone on the level of expression of IKK proteins, 30 μg whole cell extracts was run on 10% SDS-PAGE, electrotransferred and immunoblotted with indicated antibodies. FIG. 4C shows guggulsterone directly inhibits IKK activity. Whole-cell extracts were prepared from untreated and TNF (0.1 nM)-treated H1299 cells (2×10⁶/ml); 200 μg/sample whole-cell extract protein was immunoprecipitated with antibodies against IKKα and IKKβ. The immune complex was treated with the indicated concentrations of guggulsterone for 30 min at 30° C., and then a kinase assay was performed. Equal protein loading was evaluated by IKKβ.

FIGS. 5A-5C. FIG. 5A shows guggulsterone inhibited TNF-induced phosphorylation of p65. H1299 cells (2×10⁶/ml) were incubated with 50 μM guggulsterone for 4 hours and then treated with 0.1 nM TNF for different times. The cytoplasmic extracts were analyzed by Western blotting using antibodies against the phosphorylated form of p65. FIG. 5B shows guggulsterone inhibited TNF-induced nuclear translocation of p65. H1299 cells (1×10⁶/ml), was either untreated or pretreated with 50 μM guggulsterone for 4 hours at 37° C. and then treated with 0.1 nM TNF for different times. Cytoplasmic and nuclear extracts were prepared and analyzed by Western blotting using antibodies against p65. FIG. 5C shows guggulsterone inhibited TNF-induced nuclear translocation of p65. H1299 cells (1×10⁶/ml) were first treated with 50 μM guggulsterone for 4 hours at 37° C. and then exposed to 0.1 nM TNF. After cytospin, immunocytochemical analysis was performed.

FIGS. 6A-6C. FIG. 6A shows guggulsterone inhibited TNF-induced NF-κB-dependent reporter gene (SEAP) expression. H1299 cells were transiently transfected with an NF-κB-containing plasmid linked to the SEAP gene and then treated with the indicated concentrations of guggulsterone. After 24 hours in culture with 0.1 nM TNF, cell supernatants were collected and assayed for SEAP activity. Results are expressed as fold activity over the activity of the vector control. FIG. 6B shows guggulsterone inhibited NF-κB-dependent reporter gene expression induced by TNFR, TRADD, TRAF, NIK, and IKKβ. H1299 cells were transiently transfected with the indicated plasmids along with an NF-κB-containing plasmid linked to the SEAP gene and then left either untreated or treated with 50 μM guggulsterone for 4 hours. Cell supernatants were assayed for secreted alkaline phosphatase activity. Results are expressed as fold activity over the activity of the vector control. Bars indicate standard deviation. FIG. 6C shows guggulsterone inhibited TNF-induced COX2 promoter activity. H1299 cells were transiently transfected with a COX2 promoter plasmid linked to the luciferase gene and then treated with the indicated concentrations of guggulsterone. After 24 hours in culture with 0.1 nM TNF, cell supernatants were collected and assayed for luciferase activity. Results are expressed as fold activity over the activity of the vector control.

FIGS. 7A-7B. FIG. 7A shows guggulsterone inhibited COX-2, MMP-9 and VEGF expression induced by TNF. H1299 cells (2×10⁶/ml) were left untreated or incubated with 50 μM guggulsterone for 4 h and then treated with 0.1 nM TNF for different times. Whole-cell extracts were prepared, and 80 μg of the whole-cell lysate was analyzed by western blotting using antibodies against COX-2, MMP-9 and VEGF. FIG. 7B shows guggulsterone inhibited cyclin D1 and c-myc expression induced by TNF. H1299 cells (2×10⁶/ml) were left untreated or incubated with 50 μM guggulsterone for 4 hours and then treated with 0.1 nM TNF for different times. Whole-cell extracts were prepared, and 80 μg of the whole-cell lysate was analyzed by Western blotting using antibodies against cyclin D1 and c-myc.

FIG. 8 shows guggulsterone inhibited the expression of anti-apoptotic gene products IAP1, XIAP, Bfl-1/A1, bcl-2, TRAF1, cFLIP, and survivin. H1299 cells (2×10⁶/ml) were left untreated or incubated with 50 μM guggulsterone for 4 hours and then treated with 0.1 nM TNF for different times. Whole-cell extracts were prepared, and 50 μg of the whole-cell lysate was analyzed by western blotting using antibodies against IAP1, XIAP, Bfl-1/A1, bcl-2, TRAF1, cFLIP, and survivin as indicated.

FIGS. 9A-9C. FIGS. 9A-9C show guggulsterone enhances apoptosis induced by TNF and chemotherapeutic agents. FIG. 9A: KBM-5 cells (5000 cells/0.1 ml) were incubated at 37° C. with TNF, paclitaxel or doxorubicin in the presence and absence of 50 μM guggulsterone, as indicated for 72 hours, and the viable cells were assayed using MTT reagent. The results are shown as the mean±s.d. from triplicate cultures. FIG. 9B: KBM-5 cells (2×10⁶/ml) were serum starved for 24 hours and then incubated with TNF alone or in combination with guggulsterone for indicated durations, and PARP cleavage was determined by western blot analysis. FIG. 9C: KBM-5 cells (2×10⁶/ml) were serum starved for 24 hours and then incubated with TNF alone or in combination with guggulsterone as indicated for 24 hours. Cell death was determined by calcein AM based live/dead assay.

FIG. 10. shows guggulsterone inhibits TNF-induced Akt activation.

FIG. 11. shows antiproliferative effects of guggulsterone against tumor cells. Different types of tumor cells (2000 in 0.1 ml) were exposed to the indicated concentration of guggulsterone for six days and then cell viability was measured by the MTT method.

FIGS. 12A-12B. show guggulsterone effectively inhibits RANKL-induced osteoclastogenesis 24 h after stimulation. RAW 264.7 cells (1×10⁴ cells) were incubated either alone or in the presence of RANKL (5 nM), and guggulsterone (5 μM) was added at the same time. Cells were cultured for different days after RANKL treatment and stained for TRAP expression. FIG. 12A shows photographs of cells (original magnification, 100×). FIG. 12B shows multinucleated (>3 nuclei) osteoclasts were counted. Values indicate mean of total osteoclasts in triplicate cultures (error bar indicates s.d.).

FIGS. 13A-13C. Guggulsterone inhibits proliferation in U937 cells. (FIG. 13A) U937 (5000 cells/0.1 ml) were incubated at 37° C. with indicated concentrations of guggulsterone for 72 h, and the viable cells were assayed using ³H-thymidine incorporation as described herein. The results are shown as the mean±s.d. from triplicate cultures. (FIG. 13B) Guggulsterone arrests the cells at G1/S phase of the cell cycle. Serum-starved U937 cells (2×10⁶ cells/ml) were incubated in the absence or in presence of 10 μM guggulsterone for indicated times. Thereafter, the cells were washed, fixed, stained with propidium iodide, and analyzed for DNA content by flow cytometry as described herein. (FIG. 13C) Guggulsterone modulates cell cycle progression. Two million U937 cells were treated with guggulsterone (10 μM) for indicated time points and then whole cell extracts were prepared. Sixty micrograms of whole cell extracts were resolved on 10% SDS-PAGE gel, electrotransferred onto a nitrocellulose membrane, and probed for cyclin D1, cdc2, p27, p21 and GADD45. The same blots were stripped and reprobed with anti-β-actin antibody to show equal protein loading (lower panel).

FIGS. 14A-14B. (FIG. 14A) Guggulsterone inhibits the expression of anti-apoptotic gene products, Bfl-1/A1, XIAP, cFLIP, Bcl-2, BclXL, and survivin. U937 cells (2×10⁶/ml) were left untreated or incubated with 10 μM guggulsterone for different times. Whole-cell extracts were prepared, and 50 μg of the whole-cell lysate was analyzed by western blotting using antibodies against Bfl-1/A1, XIAP, cFLIP, Bcl-2, BclXL, and survivin as indicated. (FIG. 14B) Guggulsterone inhibits COX-2 and c-myc expression. U937 cells (2×10⁶/ml) were left untreated or incubated with 10 μM guggulsterone for different times. Whole-cell extracts were prepared, and 80 μg of the whole-cell lysate was analyzed by western blotting using antibodies against COX-2 and c-myc.

FIGS. 15A-15C. Guggulsterone induces apoptosis. (FIG. 15A) Cells were treated with 10 μM guggulsterone for indicated time points and then incubated with anti-annexin V antibody conjugated with FITC and analyzed with a flow cytometer for early apoptotic effects. (FIG. 15B) U937 cells were treated with different concentrations of guggulsterone for 48 h. Cells were fixed, stained with TUNEL assay reagent, and then analyzed with a flow cytometer for apoptotic effects. (FIG. 15C) Cells were treated with 10 μM guggulsterone for indicated time points. Cells were stained with Live and Dead assay reagent for 30 min and then analyzed under a fluorescence microscope. Dead cells fluoresce red and live cells fluoresce green.

FIG. 16. Guggulsterone induces caspase activation, cytochrome c release and PARP cleavage. Cells were treated with 10 μM guggulsterone for the indicated times. Whole-cell extracts were prepared and subjected to western blot analysis using antibodies against caspase 8, cytochrome C, caspase 9, caspase 3 and PARP.

FIGS. 17A-17H. (FIG. 17A) Guggulsterone inhibits Akt activation. U937 cells were incubated with 10 μM guggulsterone for the indicated times. Whole-cell extracts were prepared and analyzed by western blot analysis using anti-phospho-specific Akt. The same membrane was blotted with anti-Akt antibody. (FIG. 17B) Guggulsterone induces JNK activation. Cells were incubated with 10 μM guggulsterone for different lengths of time. Whole-cell extracts were immunoprecipitated with an antibody against JNK and analyzed with an immunocomplex kinase assay. To examine the effect of guggulsterone on the level of expression of JNK proteins, whole-cell extracts were fractionated on SDS-PAGE and examined by western blot analysis using anti-JNK antibodies. The results shown are representative of three independent experiments. (FIGS. 17C and 17D) JNK inhibition blocks caspase 3 activation. U937 cells were pretreated with 10 μM JNK inhibitor (SP600125) for 1 h and then treated with 10 μM guggulsterone for indicated times, whole cells extract were prepared and examined for JNK activation (FIG. 17C) and caspase 3 activation (FIG. 17D). (FIG. 17E) JNK inhibition blocks PARP cleavage. U937 cells were pretreated with JNK inhibitor (SP600125) for 1 h and then treated with 10 μM guggulsterone for indicated times, whole cells extract were prepared and examined for PARP cleavage. (FIG. 17F) JNK inhibition blocks guggulsterone induced cytotoxicity. U937 (5000 cells/0.1 ml) were incubated at 37° C. with indicated concentrations of guggulsterone alone or in combination with JNK inhibitor for 72 h, and the viable cells were assayed by MTT uptake. The results are shown as the mean±s.d. from triplicate cultures. (FIG. 17G) MKK4 is required for guggulsterone induced JNK activation. MKK4 mutants and wild type cells were treated with 10 μM guggulsterone for indicated time points and JNK assay was performed as described in materials and methods. (FIG. 17H) MKK4 is required for guggulsterone induced cytotoxicity. MKK4 mutants and wild type cells (1000 cells/0.1 ml) were incubated at 37° C. with indicated concentrations of guggulsterone for 72 h, and the viable cells were assayed by MTT uptake. The results are shown as the mean±s.d. of triplicate cultures.

FIGS. 18A-18C. RANKL induces NF-κB activation and guggulsterone inhibits it in dose- and time-dependent manner. (FIG. 18A) RAW 264.7 cells (1×10⁶ cells) were pre-incubated with guggulsterone (50 μM) for 4 h and then treated with 10 nM RANKL for the indicated times, and then tested for nuclear NF-κB by EMSA. (FIG. 18B) RAW 264.7 cells (1×10⁶ cells) were co-incubated without or with the indicated concentrations of guggulsterone and RANKL (10 nM) and tested for nuclear NF-κB by EMSA. (FIG. 18C) The binding of NF-κB is specific and consists of p50 and p65 subunits. Nuclear extracts were prepared from untreated RAW 264.7 cell or the cells treated with RANKL, incubated for 15 min with different Abs or unlabeled oligonucleotide probe, and then assayed for NF-κB by EMSA in 5% gel.

FIGS. 19A-19C. Guggulsterone inhibits RANKL-induced IκBα phosphorylation and degradation through inhibition of IKK activity. RAW 264.7 cells (1×10⁶ cells) were pre-incubated with guggulsterone (50 μM) for 4 h and then treated with 10 nM RANKL for the indicated times; cytoplasmic extracts were prepared to check the following: the level of IκBα (FIG. 19A); the level of phosphorylated IκBα by western blot analysis (FIG. 19B); IKK activity (FIG. 19C, upper); immunoprecipitated IKK and performed the kinase assay, and total IKK-α and IKK-β proteins by western blot analysis (FIG. 19C, middle and lower) in cytoplasmic extracts. Quantitation of IκBα after normalization with β-actin (FIG. 19A and FIG. 19B) is presented.

FIGS. 20A-20B. Guggulsterone inhibits RANKL-induced osteoclastogenesis. RAW 264.7 cells (1×10⁴ cells) were incubated either alone or in the presence of RANKL (5 nM) without or with indicated concentration of guggulsterone for 5 days and stained for TRAP expression. (FIG. 20A) TRAP-positive cells were photographed (original magnification, 100). (FIG. 20B) Multinucleated (3 nuclei) osteoclasts were counted.

FIGS. 21A-21B. Guggulsterone effectively inhibits RANKL-induced osteoclastogenesis 24 h after stimulation. RAW 264.7 cells (1×10⁴ cells) were incubated either alone or in the presence of RANKL (5 nM), and guggulsterone (5 μM) was added at the same time or after indicated time periods. Cells were cultured for 5 days after RANKL treatment and stained for TRAP expression. (FIG. 21A) Photographs of cells (original magnification, 100). (FIG. 21B) Multinucleated (3 nuclei) osteoclasts were counted.

FIGS. 22A-22B. Guggulsterone inhibits MDA-MB-468- and U266-induced osteoclastogenesis. RAW 264.7 cells (1×10⁴ cells) were incubated either alone or in the presence of MDA-MB-468 cells (1×10³ cells) or U266 cells (1×10³ cells) and guggulsterone (5 μM) was added at the same time or after indicated time periods. Cells were cultured for 5 days after co-incubation and stained for TRAP expression. (FIG. 21A) Photographs of cells (original magnification, 100). (FIG. 21B) Multinucleated (3 nuclei) osteoclasts were counted.

FIGS. 23A-23B. (FIG. 23A) U266 (2×10⁶ cells/ml) cells were treated with 25 μM guggulsterone for indicated time points as indicated and whole-cell extracts were prepared. Fifty micrograms of whole cell extracts were resolved on 7.5% SDS-PAGE, electrotransferred to a nitrocellulose membrane, and probed for the phosphorylated-STAT3 (upper panel) and stripped and reprobed for STAT3 (lower panel) (FIG. 23B) U266 (2×10⁶ cells/ml) cells were treated with indicated concentrations of guggulsterone for 4 h and whole-cell extracts were prepared. Fifty micrograms of whole cell extracts were resolved on 7.5% SDS-PAGE, electrotransferred to a nitrocellulose membrane, and probed for the phosphorylated-STAT3 (upper panel) and stripped and reprobed for STAT3 (lower panel)

FIG. 24. RPMI 8226 cells (2×10⁶ cells) were treated with 25 μM guggulsterone for indicated time points and stimulated with IL-6 (10 ng/ml) for 10 min, and whole-cell extracts were prepared. Fifty micrograms of whole cell extracts were resolved on 7.5% SDS-PAGE, electrotransferred to a nitrocellulose membrane, and probed for the phosphorylated-STAT3 (upper panel) and stripped and reprobed for STAT3 (lower panel)

### DETAILED DESCRIPTION OF THE INVENTION

The present invention provides additional compositions and methods for modulating NF-κB activity. Embodiments of the present invention include methods of administering guggulsterone for modulating the activity or activation of NF-κB. Guggulsterone may also abrogate the expression of NF-κB-regulated gene products that inhibit apoptosis, promote inflammation, and promote tumor metastasis. Guggulsterone may be derived from Commiphora mukul and is used to treat obesity, diabetes, hyperlipidemia, atherosclerosis, and osteoarthritis. Furthermore, guggulsterone has been shown to antagonize the farnesoid X receptor (FXR) and decrease the expression of bile acid-activated genes (Urizar et al., 2002; Cui et al., 2003).

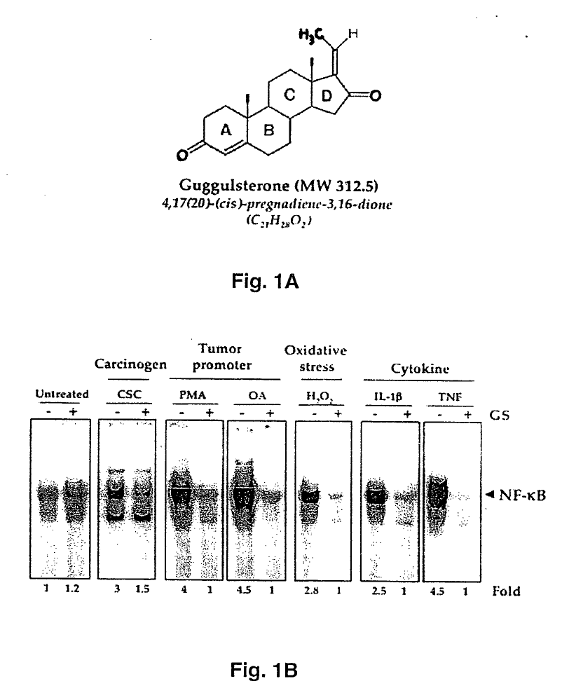

One aspect of the invention includes guggulsterone suppression of NF-κB activation induced by carcinogens and inflammatory agents. A further aspect is the regulation or modulation of NF-κB-regulated gene expression that mediates inflammation and carcinogenesis. Guggulsterone has steroid-like structure (FIG. 1A), and can be dissolved in DMSO or other know pharmaceutically acceptable delivery vehicles. In a particular aspect, guggulsterone is administered in order to modulate the cellular effects of TNF, including inflammation, tumor proliferation, tumor promotion, tumor invasion, and tumor metastasis.

In still further aspects of the invention, there is provided a method of inhibiting NF-κB activation in a cell, by contacting the cell with guggulsterone or a guggulsterone analog. In general, NF-κB activation induced by carcinogens, such as phorbol ester, okadaic acid, and cigarette smoke; by inflammatory stimuli, such as TNF, IL-1β and H₂O₂; and constitutive activation of NF-κB by administration of guggulsterone or a guggulsterone analog. In addition, guggulsterone may be used to modulate NF-κB-dependent gene transcription induced by TNF, TNFR1, TRADD, TRAF2, NIK, and IKK. Examples of genes that are directly or indirectly regulated by NF-κB, include, but are not limited to anti-apoptotic genes (e.g., IAP1, XIAP, Bfl-1/A1, bcl-2, cFLIP, survivin); proliferation related genes (cyclin D1, c-myc) and metastasis related genes (e.g., MMP-9, COX2 and VEGF).

In yet still another aspect of the invention, there is provided a method of increasing the effect of apoptosis-inducing agent in a cell by contacting the cell with both an apoptosis inducing agent and guggulsterone or an analog thereof. In particular embodiments, the apoptosis inducing agent can be TNF, a chemotherapeutic agent such as paclitaxel or doxorubicin, and/or an antiproliferative or anticancer therapy. The inventive composition will be administered in amount effective to result in amelioration of an inflammatory, hyperproliferative, or cancerous state. Typically, at least a betterment of the patients quality of life will be effected. In particular embodiments, guggulsterone or a guggulsterone analog can be used to treat cancer in an individual.

In still yet another aspect of the invention, there is provided a method of inhibiting NF-κB activated osteoclastogenesis in a cell, by contacting the cell with guggulsterone or a guggulsterone analog. Inhibition of osteoclastogenesis will aid in ameliorating conditions associated with bone loss or resorption.

It is specifically contemplated that methods of the present invention utilize pharmaceutically acceptable compositions comprising guggulsterone or a guggulsterone analog. In view of the published clinical trials and other studies involving the use of guggulsterone or a guggulsterone analog, a person having ordinary skill in this art would readily be able to determine appropriate dosages and routes of administration of guggulsterone or a guggulsterone analog. When used in vivo for therapy, guggulsterone or a guggulsterone analog is administered to the patient or an animal in therapeutically effective amounts, for example in amounts that inhibit NF-κB activation, induce apoptosis in cancer cells, inhibit osteoclastogenesis, and/or enhance the effectiveness of other therapeutic agents.

I. Guggulsterones

Ayurveda, a philosophy and healing system developed over thousands of years in India that employs botanical preparations, takes a holistic view of human disease. It views any disease as a dysfunction of the whole body rather than of a single organ or physiological process. Most of the Ayurvedic drugs therefore are likely to act on a number of dysfunctions of the body involving a number of organs and functions. Gugulipid, an ethylacetate soluble fraction of gum guggul, was developed as a hypolipidemic agent, based on the reference to the lipid lowering effect of guggul resin in Charak Samhita, a classic text of Ayurveda. Chemopharmacological investigation of this extract resulted in the characterization of guggulsterone [cis-and trans-4,17(20)-pregnadiene-3,16-dione] as the major constituent. Guggulsterone may be isolated from a gum/resin of the plant Commiphora mukul or Commiphora wightii. Guggal contains a complex mixture of terpenes, sterols, esters and higher alcohols. The ethyl acetate extract of the resin is an oily resinous material known as “gugulipid” or “guggal lipid.” For additional information regarding guggal and/or guggulsterones see U.S. Pat. Nos. 6,896,901; 6,086,889; 6,737,442; 6,630,177; 6,436,991; 6,277,396; 6,120,779; 6,113,949; 6,019,975; 5,972,341; 5,690,948; and 5,273,747, each of which are incorporated in their entirety by reference.

NF-κB activation has been found in most inflammatory diseases, it is postulated that guggulsterone modulates NF-κB activation. As described herein, guggulsterone suppresses NF-κB activation by carcinogens (phorbol ester, okadaic acid, cigarette smoke) and inflammatory stimuli (hydrogen peroxide, TNF and interleukin 1β) through inhibition of IKK, IκBα phosphorylation and degradation, which lead to abrogation of p65 phosphorylation and nuclear translocation. NF-κB-dependent reporter gene transcription induced by TNF, TNFR1, TRADD, TRAF2, NIK and IKK, was also blocked. The expression of anti-apoptotic genes (IAP1, XIAP, Bfl-1/A1, bcl-2, cFLIP, survivin), proliferative genes (cyclin d1, c-myc) and metastasis related genes (MMP-9, COX2 and VEGF) are also downregulated by guggulsterone.

There is little known about the mechanism of action of guggulsterone. The results presented herein clearly indicate that guggulsterone can suppress NF-κB activation induced by a wide variety of agents. This suggests that the site of action of guggulsterone is common to all these agents. IKK was identified as a target site for the action of guggulsterone. Cells that were exposed to guggulsterone failed to activate IKK in response to TNF. Surprisingly, incubation of IKK with guggulsterone was sufficient to suppress its activity. These results show that guggulsterone is a direct inhibitor of IKK. This is the first report to suggest that a steroid can suppress NF-κB activation through inhibition of IKK activity. Although steroids are known to exhibit anti-inflammatory activity, whether it is through suppression of NF-κB, is less clear. Auphan et al. showed that glucocorticoids inhibited NF-κB activation and this inhibition is mediated through induction of the IκBα, which traps activated NF-κB in inactive cytoplasmic complexes (Auphan et al., 1995). Interestingly, estradiol was found to activate NF-κB (Shyamala et al., 1992).

By NF-κB gene reporter assay, it is shown that guggulsterone suppresses NF-κB activation induced by TNF, TNFR1, TRADD, TRAF2, NIK and IKK, but not that activated by p65. Although this pathway may be restricted to TNF-induced NF-κB activation, these results confirm that IKK is a potential target of guggulsterone. Guggusterone may suppress NF-κB activation through other biological mechanisms as well.

Various tumor cell types express constitutively active NF-κB, the mechanism of which is not fully understood, and it is critical for their proliferation (Garg et al., 2002; Bharti et al., 2003; Giri et al., 1998). Potential mechanisms responsible for constitutive activation include overexpression of IκBα; mutations in the IκBα gene (Emmerich et al., 1999); enhanced IκBα degradation (Miyamoto et al., 1994); constitutive expression of TNF (Giri et al., 1998); and constitutive expression of IL-1 (Estrov et al., 1999). Guggulsterone suppresses constitutive NF-κB activity in human multiple myeloma cells and head and neck squamous cell carcinoma cells.

It has been established that activation of NF-κB regulates genes that control proliferation and metastasis of cancer (Karin et al., 2002; Garg et al., 2002; Huang et al., 2000). The results presented herein demonstrate that guggulsterone can suppress the expression of COX2, MMP-9, VEGF, cyclin D1 and c-myc, all regulated by NF-κB. Thus, guggulsterone may suppress proliferation of tumor cells and their metastasis.

Guggulsterone suppresses the expression of numerous anti-apoptotic gene products, all known to be regulated by NF-κB activation. The overexpression of IAP1, XIAP, Bfl-1/A1, bcl-2, cFLIP, and survivin has been found in numerous tumors and has been linked to survival, chemoresistance and radioresistance. Since most of these gene products are downregulated by guggulsterone, it is contemplated that guggulsterone will aid in overcoming or reducing radioresistance and/or chemoresistance. In addition, guggulsterone can potentiate the apoptotic effects of TNF and chemotherapeutic agents, such as paclitaxel and doxorubicin. Thus guggulsterone which is a pharmacologically safe agent (Urizar et al., 2003), can be used as an anticancer agent either alone or in combination with existing anti-cancer therapies.

Cancer is a hyperproliferative disorder characterized by the upregulation of genes responsible for transformation, proliferation, invasion, angiogenesis and metastasis. A number of these activities are influenced by the aberrant activity of NF-κB. Therefore, in one embodiment of the invention includes methods of using guggulsterone to suppress NF-κB activation, in particular NF-κB activation induced by various carcinogens, inflammatory agents, and tumor promoters.

II. Bone Absorption-Resorption Cycle

NF-κB has been implicated in modulating osteoclastogenesis, an integral part of bone absorption-resorption cycle. Living bone tissue is continuously being replenished by the process of resorption and deposition of calcium minerals. This process, described as, the absorption-resorption cycle, is facilitated by two cell types, the osteoblasts and the osteoclasts. The osteoclast is a multinucleated cell and is the only cell in the body known to have the capacity to degrade (or resorb) bone. This resorption activity is accomplished by the osteoclast forming pits (resorption lacunae) in bone tissue. In fact, osteoclast activity in cell culture is measured by their capacity to form these pits on slices of mineralized tissue such as bone or sperm whale dentine. The osteoclast is derived from a hematopoietic precursor which it shares with the formed elements of the blood (Takahashi et al., 1987). The precursor for the osteoclast is a mononuclear cell (cell with a single nucleus) which is found in the bone marrow and which forms the mature and unique multinucleated osteoclast after undergoing replication and differentiation by means of cell fusion. The mature osteoclast is distinguished from other multinucleated cells by the presence of the enzyme tartrate-resistant acid phosphatase (TRAP) which is often used as an osteoclast cell marker.

Among the pathological conditions associated with an abnormal osteoclast development or function are conditions wherein increased bone resorption results in the development of fragile and/or brittle bone structure, such as osteoporosis, or increased bone absorption results in the development of excess bone mass, such as osteopetrosis. It is believed that the development of excess or deficient populations of osteoclasts or osteoblasts results from a corresponding lack or excess of specific cytokines.

Knockout mouse models of RANKL, RANK, and osteoprotegerin decoy receptor (OPG) have demonstrated an essential role of these molecules in osteoclastogenesis. The biological importance of these molecules is underscored by the induction of severe osteoporosis by targeted disruption of OPG and by the induction of osteopetrosis by targeted disruption of RANKL or by overexpression of OPG (Bucay et al., 1998; Kong et al., 1999; Mizuno et al., 1998). Thus, osteoclast formation may be attributed to the relative ratio of RANKL to OPG in the microenvironment of bone marrow, and alterations in this balance may be a major cause of bone loss in many metabolic bone disorders. Similar to RANKL −/− mice, targeted disruption of RANK also leads to an osteopetrotic phenotype (Dougall et al., 1999; Li et al., 2000). Both RANK −/− and RANKL −/− mice exhibited absence of osteoclasts, indicating the essential requirement of these molecules for osteoclastogenesis. Furthermore, RANK and RANKL are required for lymph node organogenesis and early B and T cell development (Dougall et al., 1999; Kong et al., 1999). Additionally, mice lacking TRAF6 (Lomaga et al., 1999), c-Src (Soriano et al., 1991), c-Fos (Johnson et al., 1992), or the NF-κB subunits p50/p52 (Franzoso et al., 1997; Iotsova et al., 1997) also display an osteopetrotic phenotype; though these mutant mice have osteoclasts, these cells apparently have defects in bone resorption.

Maintenance of bone integrity requires a dynamic balance between bone formation and bone resorption. The net pool size of active osteoclasts is determined by the net effects of differentiation and fusion of osteoclast precursors and by the activity and rate of apoptosis of active osteoclasts. Although various cytokines (TNF, IL-1, IL-6, IL-11, TGFα) and molecules (11α, 25-dihydroxyvitamin D3 and glucocorticoids) expressed by osteoblast lineage cells have been shown to play a role in osteoclast differentiation, it appears that the essential factors are RANKL (produced by osteoblasts) and RANK (expressed on osteoclasts and osteoclast progenitors), as well as the resultant intracellular signaling mechanisms and pathways. There is also a requirement for M-CSF, but its function still remains elusive, but is probably only required for the initiation of differentiation of the early osteoclast progenitors and survival.

Excessive bone resorption by osteoclasts contributes to the pathology of many human diseases including arthritis, osteoporosis, periodontitis, and hypercalcemia of malignancy. During resorption, osteoclasts remove both the mineral and organic components of bone (Blair et al., 1986). The current major bone diseases of public concern are osteoporosis, hypercalcemia of malignancy, osteopenia due to bone metastases, periodontal disease, hyperparathyroidism, periarticular erosions in rheumatoid arthritis, Paget's disease, immobilization-induced osteopenia, and glucocorticoid treatment. All these conditions are characterized by bone loss, resulting from an imbalance between bone resorption (breakdown) and bone formation, which continues throughout life at the rate of about 14% per year on the average. However, the rate of bone turnover differs from site to site, for example it is higher in the trabecular bone of the vertebrae and the alveolar bone in the jaws than in the cortices of the long bones. The potential for bone loss is directly related to turnover and can amount to over 5% per year in vertebrae immediately following menopause, a condition which leads to increased fracture risk. Turnover may be effected by an increase or decrease in osteoblast activity or an increase or decrease in osteoclast activity. Compositions and methods for modulating osteoclast and/or osteoblast in a subject would be useful in the treatment of a variety of diseases or conditions associated with bone loss. All of these conditions may benefit from treatment with agents which inhibit or regulate osteoclastogenesis or bone resorption.

III. Methods for Modulation of NF-κB

The present invention involves methods and compositions for the modulation of NF-κB. In particular aspects, the methods and compositions of the invention may be used in the treatment of subjects with, suspected of having, or having a propensity of developing hyperproliferative diseases or conditions, pathologic bone loss, or any combination thereof. The term “therapeutic benefit” refers to anything that promotes or enhances the well-being of the subject with respect to the medical treatment of his/her condition, which includes treatment of hyperproliferative conditions and/or regulation of osteoclastogenesis.

A. Pharmaceutical Formulations and Delivery

In certain embodiments of the present invention, methods involving delivery of one or more compounds of the invention, guggulsterone or analogs thereof, are contemplated. Examples of diseases and conditions that may be prevented, ameliorated, or treated with one or more compound of the invention include cancer, which includes, but is not limited to lung, head and neck, breast, pancreatic, prostate, renal, bone, testicular, cervical, gastrointestinal, colon, bladder and other cancers.

An “effective amount” of the pharmaceutical composition, generally, is defined as that amount sufficient to detectably and repeatedly to achieve the stated desired result, for example, to ameliorate, reduce, minimize or limit the extent of the hyperproliferative or osteolytic condition or disease, or its symptoms. In certain aspects, more rigorous definitions may apply, including prevention, elimination, eradication or cure of disease.

In certain specific embodiments, it is desired to treat cancer, inhibit osteoclastogenesis or both, as well as otherwise reverse, hinder, or reduce the size of a cancerous lesion, or resorption of bone using the methods and compositions of the present invention. The routes of administration will vary, naturally, with the location and nature of the lesion and may include, for example, intradermal, subcutaneous, regional, parenteral, intravenous, intramuscular, intranasal, systemic, and oral administration and formulation.

Continuous administration also may be applied where appropriate. Delivery via syringe or catheterization is contemplated. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection or administration but may comprise continuous infusion over a set period of time. Unit dose of the present invention may conveniently be described in terms of milligram (mg) per volume of formulation, weight of therapeutic composition, or weight of subject to be treated.

In some embodiments, the method for the delivery of a composition comprising one or more compositions of the invention is via systemic administration. However, the pharmaceutical compositions disclosed herein may alternatively be administered parenterally, subcutaneously, intratracheally, intravenously, intradermally, intramuscularly, or even intraperitoneally. Injection may be by syringe or any other method used for injection of a solution.

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi.

The carrier can be a solvent or dispersion medium containing, for example, water, ethanol, DMSO, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the some methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

B. Combination Treatments

In certain embodiments, the compositions and methods of the present invention involve a compound inhibiting NF-κB activation, treating hyperproliferative conditions, treating cancer, inhibiting growth, inducing apoptosis, enhancing apoptosis, regulating bone resorption, inhibiting osteoclast activity, inhibiting osteoclastogenesis, or combinations thereof which in turn may be used in combination with other agents or compositions to enhance the effect of other treatments, such as anti-neoplatic treatments, to better the quality of life of a subject being treated. These compositions would be provided in a combined amount effective to achieve the desired effect, for example, inhibiting or modulating inflammation, modulating or treating growth hyperproliferative cells, killing or growth inhibition of a cancer cell, and inhibition of osteoclasotgenesis, the activity of osteoclasts, or the resorption of bone. This process may involve contacting the cells with a composition of the invention, and a second therapeutic agent(s) or multiple factor(s) at the same time. This may be achieved by contacting the cell with a single composition or pharmacological formulation that includes two or more agents, or by contacting the cell with two or more distinct compositions or formulations wherein at least one composition includes a composition of the invention and one or more other compositions includes at least a second therapeutic agent.

In one embodiment of the present invention, it is contemplated that anti-cancer, anti-osteoclast, or both anti-cancer and anti-osteoclast therapy is used in conjunction with immune therapy intervention, in addition to pro-apoptotic, anti-angiogenic, anti-cancer, or cell cycle regulating agents. Alternatively, the guggulsterone therapy may precede or follow treatment using another agent or therapy by intervals ranging from minutes to weeks. In embodiments where one or more second therapeutic agent and guggulsterone therapy are applied separately to a cell, tissue, organ or subject, one would generally ensure that a significant period of time did not expire between the time of each delivery, such that the second agent and the inventive composition would still be able to exert an advantageously combined effect on the subject. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, for example a composition of the present invention is “A” and a second therapy, such as chemotherapy, is “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the guggulsterone compositions of the present invention to a patient will follow general protocols for the administration of such compositions, taking into account the toxicity, if any. It is expected that the treatment cycles would be repeated as necessary. It also is contemplated that various standard therapies, as well as surgical intervention, may be applied in combination with the described therapy.

In specific embodiments, it is contemplated that an anti-cancer therapy, such as chemotherapy, radiotherapy, or immunotherapy, is employed in combination with the guggulsterone therapies described herein.

1. Chemotherapy

Cancer therapies include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves, proton beam irradiation (U.S. Pat. No. 5,760,395 and U.S. Pat. No. 4,870,287) and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic composition and a chemotherapeutic or radiotherapeutic agent are delivered to a target cell, tissue or subject, or are placed in direct juxtaposition.

3. Immunotherapy

In the context of cancer treatment, immunotherapeutics, generally, rely on the use of immune effector cells and molecules (e.g., monoclonal antibodies) to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993). In melanoma immunotherapy, those patients who elicit high IgM response often survive better than those who elicit no or low IgM antibodies (Morton et al., 1992). IgM antibodies are often transient antibodies and the exception to the rule appears to be anti-ganglioside or anticarbohydrate antibodies.

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated anigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders were few compared to those who did not respond.

4. Surgery

Approximately 60% of persons with cancer will undergo surgery of some type, which includes preventative, diagnostic or staging, curative and palliative surgery. Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

IV. STAT-3 Pathways

Numerous reports suggest that interleukin-6 (IL-6) promotes survival and proliferation of multiple myeloma (MM) cells through the phosphorylation of a cell signaling protein, STAT3. Thus agents that suppress STAT3 phosphorylation are contemplated for the treatment of MM. Guggulsterone, which has been demonstrated to be a pharmacologically safe agent in humans, inhibited IL-6-induced STAT3 phosphorylation and consequent STAT3 nuclear translocation. The constitutive phosphorylation of STAT3 found in certain MM cells was also abrogated by treatment with guggulsterone. Overall, studies have demonstrated that guggulsterone was a potent inhibitor of STAT3 phosphorylation. All methods and compositions described herein are contemplated to be used in conjunction with inhibition of STAT-3 activity.

Multiple myeloma (MM) is a B cell malignancy characterized by the latent accumulation in bone marrow of secretory plasma cells with a low proliferative index and an extended life span (Hallek et al., 1998). MM accounts for 1% of all cancers and >10% of all hematologic cancers. Agents used to treat myeloma includes combinations of vincristine, BCNU, melphalan, cyclophosphamide, Adriamycin, and prednisone or dexamethasone (Forum, 2001). Usually, patients younger than 65 years are treated with high-dose melphalan with autologous stem-cell support, and older patients who cannot tolerate such intensive treatment receive standard-dose oral melphalan and prednisone. Despite these treatments, only 5% of patients achieve complete remission and the median survival is only 30-36 months (1973-1989 Annual Cancer Statistics Review. National Cancer Institute, Bethesda, 1992; Feinman et al., 1999).

The dysregulation of the apoptotic mechanism in plasma cells is considered a major underlying factor in the pathogenesis and subsequent chemoresistance in MM. It is established that IL-6, produced in either an autocrine or paracrine manner, has an essential role in the malignant progression of MM by regulating the growth and survival of tumor cells (Kawano et al., 1988; Klein et al., 1995). IL-6 induces intracellular signaling through a member of the signal transducers and activators of transcription (STAT) family. Engagement of cell surface cytokine receptors activates the Janus kinase (JAK) family of protein tyrosine kinases, which phosphorylate and activate cytoplasmic STAT proteins (Darnell, 1997; Taga and Kishimoto, 1997). Activated STATs dimerize and translocate to the nucleus, where they bind to specific DNA response elements and induce expression of STAT-regulated gene expression. One STAT family member, STAT3, has been described in mediating the IL-6 signaling through interaction with the IL-6 receptor, and studies using dominant-negative STAT3 proteins have demonstrated a requirement of STAT3 signaling in tumor transformation (Bromberg et al., 1998; Turkson et al., 1998). Evidence is accumulating that constitutive activation of STAT3 proteins occurs frequently in human tumor cells (Garcia and Jove. 1998; Garcia et al., 1997; Weber-Nordt et al., 1996; Takemoto et al., 1997), implicating aberrant STAT3 signaling as an important process in malignant progression. Recently, Catlett-Falcone et al. have shown that human MM cells also express constitutively activated STAT3, which confers resistance to apoptosis in these cells through expression of high levels of the anti-apoptotic protein Bcl-xL (Catlett-Falcone et al., 1999; Tu et al., 1998; Grad et al., 2000). Bcl-2 overexpression, another important characteristic of most MM cell lines (Pettersson et al., 1992), rescues these tumor cells from chemotherapy-induced apoptosis (Feinman et al., 1999; Tu et al., 1996).

Thus pharmacologically safe and effective agents that can block constitutive or inducible activation of STAT3 as treatments have a potential for MM and other diseases.

### EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the cells and methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

A. Material and Methods

Reagents: Z-Guggulsterone, obtained from Steraloids, Inc. (Newport, R.I.), was dissolved in dimethyl sulfoxide (DMSO) as a 100 mM stock solution and stored at −20° C. Bacteria-derived human TNF, purified to homogeneity with a specific activity of 5×10⁷ U/mg, was provided by Genentech Inc. (South San Francisco, Calif.). Penicillin, streptomycin, RPMI 1640 medium, FBS and lipofectamine 2000 were obtained from Invitrogen (Grand Island, N.Y.). The following polyclonal antibodies were obtained from Santa Cruz Biotechnology, Inc. (Santa Cruz, Calif.): anti-p65, against the epitope corresponding to amino acids mapping within the amino terminal domain of human NF-κB p65; anti-p50, against a peptide 15 amino acids long mapping at the nuclear localization sequence region of NF-κB p50; anti-IκBα, against amino acids 297-317 mapping at the carboxyl terminus of IκBα/MAD-3, and anti-c-Rel and anti-cyclin D1 against amino acids 1-295, which represents full-length cyclin D1 of human origin. Phospho-IκBα (Ser32) antibody was purchased from New England BioLabs (Beverly, Mass.). Antibodies of anti-cyclin D1, anti-MMP-9, anti-polyadenosine ribose polymerase (PARP), anti-IAP1, anti-IAP2, anti-Bcl-2, anti-Bfl-1/A1, and anti-TRAF1 were obtained from Santa Cruz Biotechnology (Santa Cruz, Calif.); anti-COX-2, anti-MMP9 and anti-XIAP antibodies from BD Biosciences (San Diego, Calif.); phospho-specific anti-IκBα (Ser32) antibody from Cell Signaling (Beverly, Mass.). Anti-IKKα and anti-IKKβ antibodies were kindly provided by Imgenex (San Diego, Calif.). Anti-Akt antibodies were obtained from Santa Cruz Biotechnology (Santa Cruz, Calif.). Phospho-specific anti-Akt antibodies were purchased from Cell Signaling (Beverly, Mass.).

Cell Lines and Cell Culture: The cell lines included human non-small cell lung carcinoma (H1299) cells, and human lung epithelial cell carcinoma (A549) cells, all provided by Dr. Reuben Lotan (The University of Texas M. D. Anderson Cancer Center). Human leukemia (Jurkat), and myelogenous leukemia (KBM-5) cells were obtained from the American Type Culture Collection (Manassas, Va.). A549, Jurkat and H1299 cells were cultured in RPMI 1640 medium, and KBM-5 cells were cultured in Iscove's modified DMEM, all supplemented with 10% FBS, 100 U/ml penicillin, and 100 μg/ml streptomycin except KBM-5 that was supplemented with 15% FBS.

NF-κB Activation: To determine NF-κB activation, EMSA was carried out as previously described (Chaturvedi et al., 2000). Briefly, nuclear extracts prepared from cells (2×10⁶/ml) treated with TNF were incubated with ³²P-end-labeled 45-mer double-stranded NF-κB oligonucleotide (8 μg of protein with 16 mol of DNA) from the human immunodeficiency virus long terminal repeat, 5′-TTGTTACAAGGGACTTTCCGCTGGGGACTTTCCAGGGA GGCGTGG-3′ (SEQ ID NO:1) (boldface indicates NF-κB binding sites), for 15 min at 37° C. The DNA-protein complex formed was separated from free oligonucleotide on 6.6% native polyacrylamide gels. A double-stranded mutated oligonucleotide, 5′-TTGTTACAACTCACTTTCCGCTGCTCACTTTCCAGGGAGGCGTGG-3′ (SEQ ID NO:2), was used to examine the specificity of binding of NF-κB to the DNA. The specificity of binding was also examined by competition with the unlabeled oligonucleotide. For supershift assays, nuclear extracts prepared from TNF-treated cells were incubated with antibodies against either the p50 or the p65 subunit of NF-κB for 30 min at room temperature before the complex was analyzed by EMSA. The dried gels were visualized, and radioactive bands were quantitated using a PHOSPHORIMAGER™ (Molecular Dynamics, Sunnyvale, Calif.) using IMAGEQUANT™ software.

Western Blot Analysis: To determine the effect of guggulsterone on TNF-dependent IκBα phosphorylation, IκBα degradation, p65 translocation and p65 phosphorylation, cytoplasmic extracts were prepared as previously described (Majumdar et al., 2001) from H1299 (2×10⁶/ml) pretreated with 50 μM guggulsterone for 4 h and then exposed to 0.1 nM TNF for various times. Thirty micrograms of cytoplasmic protein was resolved on 10% SDS-PAGE gel, transferred to a nitrocellulose membrane, blocked with 5% non-fat milk, and probed with specific antibody against IκBα, posphorylated IκBα, p65 and phosphorylated p65. To determine the expression of cyclin D1, COX-2, MMP-9, clAP-1, xlAP, TRAF1, Bcl-2, Bfl-1, cFLIP, and survivin, whole-cell extracts were prepared from treated cells (2×10⁶ cells in 2 ml medium), 30-50 μg protein was resolved on SDS-PAGE gel and probed by Western blot with specific antibodies as per manufacturer's recommended protocol. The blots were washed, exposed to HRP-conjugated secondary antibodies for 1 hour, and finally detected by ECL reagent (Amersham Pharmacia Biotech.). The bands were quantitated using a Personal Densitometer Scan v1.30 using IMAGEQUANT™ software version 3.3 (Molecular Dynamics).

Immunolocalization of NF-κB p65: The effect of guggulsterone on CS-induced nuclear translocation of p65 was examined using an immunocytochemical method as described previously (Bharti et al., 2003). Stained slides were mounted with mounting medium (Sigma Chemical) and analyzed under an epifluorescence microscope (Labophot-2; Nikon, Tokyo, Japan). Images were captured using a PHOTOMETRICS COOLSNAP CF™ color camera (Nikon, Lewisville, Tex.) and METAMORPH™ version 4.6.5 software (Universal Imaging, Downingtown, Pa.).

IKK Assay: To determine the effect of guggulsterone on TNF-induced IKK activation, IKK was analyzed by a method essentially as described previously (Shishodia et al., 2003). Briefly, IKK complex was precipitated from whole-cell extracts with antibody to IKKα and IKKβ, assayed in kinase assay using GST-IκBα (1-54) as a substrate, and resolved on 10% polyacrylamide gel; the radioactive bands were visualized using a PHOSPHORIMAGER™. To determine the total amounts of IKKα and IKKβ in each sample, 30 μg of the whole-cell extract protein was resolved on a 7.5% polyacrylamide gel and analyzed by Western blot using antibody against IKKα and IKKβ.

NF-κB-dependent Reporter Gene Transcription: The effect of guggulsterone on TNF-induced NF-κB dependent reporter gene transcription was measured as previously described (Manna et al., 2000). Briefly, H1299 cells (5×10⁵ cells/well) were plated in six-well plates and transiently transfected the next day by LIPOFECTAMINE™ 2000 method with pNF-κB-SEAP using the manufacturer's protocol. To examine TNF-induced reporter gene expression, cells was transfected with 2 μg of the SEAP expression plasmid. After 18 hours, medium was removed, cells were rinsed and treated with various doses of guggulsterone for 4 hours and then treated with TNF. Twenty four hours later, the cell culture medium was harvested and analyzed for alkaline phosphatase (SEAP) activity essentially according to the protocol described by the manufacturer (Clontech, Palo Alto, Calif.) using a 96-well fluorescence plate reader (FLUOROSCAN™ II; Labsystems, Chicago, Ill.) with excitation set at 360 nm and emission set at 460 nm.

COX-2 Promoter-dependent Reporter Luciferase Gene Expression: COX-2 promoter activity was examined as described elsewhere (Shishodia et al., 2003). To further determine the effect of guggulsterone on COX-2 promoter, A293 cells were seeded at a concentration of 1.5×10⁵ cells per well in six-well plates. After overnight culture, the cells in each well were transfected with 2 μg DNA consisting of COX-2 promoter-luciferase reporter plasmid, along with 6 μl of LIPOFECTAMINE™ 2000 (Life Technologies, Inc.) according to the manufacturer's protocol. The COX-2 promoter (−375 to +59), amplified from human genomic DNA by using the primers 5′-GAGTCTCTTATTTATTTTT-3′ (sense) (SEQ ID NO:3) and 5′-GCTGCTGAGGAGTTCCTGGACGTGC-3′ (antisense) (SEQ ID NO:4), was provided by Dr. Xiao-Chun Xu (M.D. Anderson Cancer Center). After a 6 hour exposure to the transfection mixture, the cells were incubated in medium containing guggulsterone for 12 hours. The cells were exposed to TNF (0.1 nM) for 24 hours and then harvested. Luciferase activity is measured by using the Promega luciferase assay system according to the manufacturer's protocol and detected by using MONOLIGHT™ 2010 (Analytical Luminescence Laboratory, San Diego, Calif.). All experiments were performed in triplicate and repeated at least twice to prove their reproducibility.

Cytotoxicity Assay: Cytotoxicity was assayed by the modified tetrazolium salt 3-(4-5-dimethylthiozol-2-yl)2-5-diphenyl-tetrazolium bromide (MTT) assay (Bharti et al., 2003). Briefly, cells (5000 cells/well) were incubated in the presence or absence of the indicated test sample in a final volume of 0.1 ml for 72 hours at 37° C. Thereafter, 0.025 ml of MTT solution (5 mg/ml in PBS) was added to each well. After a 2 hour incubation at 37° C., 0.1 ml of the extraction buffer (20% SDS, 50% dimethylformamide) was added. After an overnight incubation at 37° C., the optical densities at 570 nm were measured using a 96-well multiscanner autoreader (Dynatech MR5000), with the extraction buffer as blank. The percent cytotoxicity was determined as follows: (1−(A₅₇₀ of test sample)/(A₅₇₀ of control sample)×100%.

PARP cleavage assay: For detection of cleavage products of PARP, whole-cell extracts were prepared by subjecting the guggulsterone-treated cells to lysis in lysis buffer (20 mM Tris, pH 7.4; 250 mM NaCI; 2 mM EDTA, pH 8.0; 0.1% Triton-X 100; 0.01 μg/ml aprotinin; 0.005 μg/ml leupeptin; 0.4 mM PMSF; and 4 mM NaVO₄). Lysates will be spun at 14000 rpm for 10 min to remove insoluble material, resolved by 10% SDS PAGE, and probed with PARP antibodies. PARP is cleaved from the 116-kDa intact protein into 85-kDa and 40-kDa peptide products. To detect cleavage products of procaspase-3 and procaspase-9, whole-cell extracts will be resolved by 10% SDS PAGE and probed with appropriate antibodies.

Live and Dead Assay: To measure apoptosis, the Live and Dead assay (Molecular Probes) was used, which determines intracellular esterase activity and plasma membrane integrity. This assay employs calcein, a polyanionic dye, which is retained within the live cells and provides green fluorescence. It also employs the ethidium monomer dye (red fluorescence), which can enter the cells only through damaged membranes and bind to nucleic acids but is excluded by the intact plasma membrane of live cells. Briefly, 1×10⁵ cells are incubated with 10 μM guggulsterone for 24 h and then treated with 1 nM TNF for 16 hours at 37° C. Cells are stained with the Live and Dead reagent (5 μM ethidium homodimer, 5 μM calcein-AM) and then incubated at 37° C. for 30 min. Cells were analyzed under a fluorescence microscope (Labophot-2).

Osteoclast differentiation assay: RAW 234.7 cells were cultured in 24-well dishes at a density of 1×10⁴ cells per well and were allowed to adhere overnight. Medium was then replaced and the cells were treated with 5 nM (˜100 ng/ml) RANKL. At different days, cultures were stained for TRAP expression using an acid phosphatase kit, and the total number of TRAP-positive multinucleated osteoclasts (>3 nuclei) per well were counted.

B. Results

Guggulsterone Blocks NF-κB Activation Induced by TNF, IL-1β, PMA, H₂O₂. Cigarette Smoke, and Okadaic Acid: TNF, IL-1b, PMA, H₂O₂, cigarette smoke and okadaic acid have been shown to activate NF-κB. Thus, the effect of gugulsterone on the activation of NF-κB by these agents in H1299 cells was investigated. As examined by DNA binding assay (EMSA), guggulsterone suppressed the NF-κB activation induced by all these agents (FIG. 1B). These results suggest that guggulsterone acts at a step in the NF-κB activation pathway that is common to all these agents.

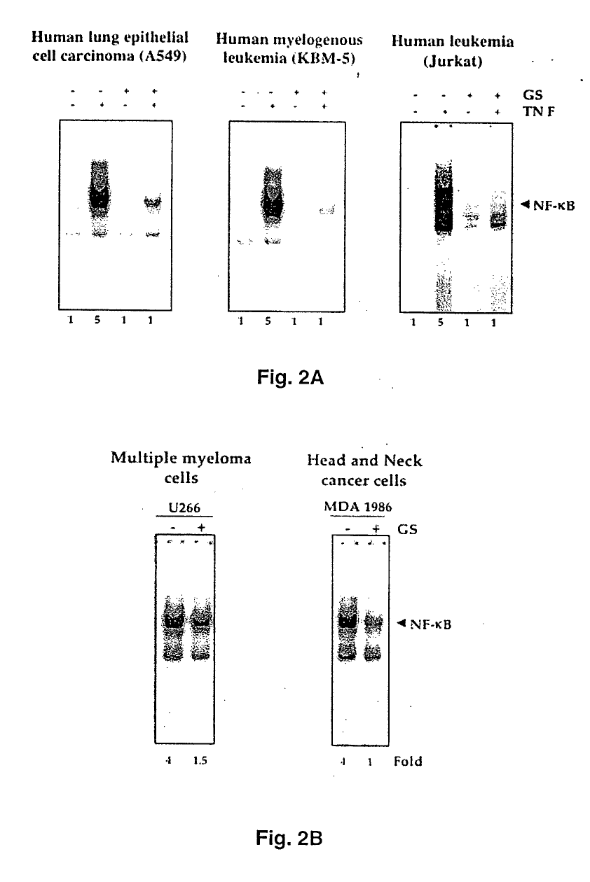

Inhibition of NF-κB Activation by Guggulsterone is Not Cell Type Specific: Some reports suggest that distinct signal transduction pathways mediate NF-κB induction in epithelial and lymphoid cells (Bonizzi et al., 1997). The effect of guggulsterone on TNF-induced NF-κB activation in lung epithelial cell carcinoma (A549), T cell leukemia (Jurkat) and myeloid leukemia (KBM-5) cells was examined. TNF activated NF-κB in all the cell types and guggulsterone completely inhibited this activation (FIG. 2A), indicating a lack of cell type specificity.

Guggulsterone Inhibits Constitutive NF-κB Activation: Certain tumor cells express constitutively active NF-κB through a mechanism which is not completely understood (Bharti et al., 2003; Giri et al., 1998). Human multiple myeloma (U266) and head and neck squamous cell carcinoma (MDA 1986) are known to express constitutive active NF-κB. Whether guggulsterone could inhibit constitutively active NF-κB in these two different cell types, was assessed. Guggulsterone completely inhibited this constitutively active NF-κB (FIG. 2B).

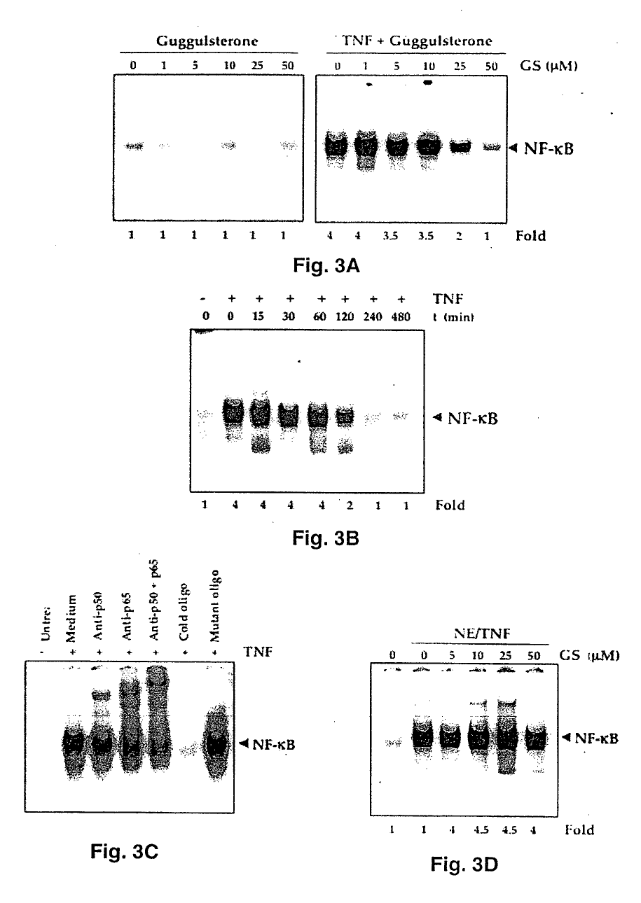

The Suppression of NF-κB by Guggulsterone is Dose- and Time-dependent: Human non-small cell lung adenocarcinoma H1299 cells were pre-incubated with different concentrations of guggulsterone and then treated with TNF. In nuclear extracts, guggulsterone, even up to 50 μM, by itself did not activate NF-κB, TNF activated NF-κB by four-fold, and guggulsterone inhibited TNF-mediated NF-κB activation in a dose-dependent manner, with maximum inhibition occurring at 50 μM (FIG. 3A). The minimum time required for complete inhibition of NF-κB activation was observed at 4 hours (FIG. 3B).

To determine whether the NF-κB bound to DNA indeed consist of p50 and p65, nuclear extracts from TNF-activated cells was incubated with antibodies to the p50 (NF-κB1) and the p65 (RelA) subunit of NF-κB. Both antibodies shifted the band to a higher molecular mass, thus suggesting that the TNF-activated complex consisted of p50 and p65 (FIG. 3C). Neither preimmune serum nor irrelevant antibody had any effect. Addition of excess unlabeled NF-κB (cold oligo; 100-fold) caused complete disappearance of the band, whereas mutated oligo had no effect on the DNA-binding.

Binding of NF-κB to the DNA is Not Directly Affected by Guqgelsterone: To determine whether guggulsterone suppresses NF-κB activation by directly modifying NF-κB proteins, as reported with TPCK (the serine protease inhibitor), herbimycin A (protein tyrosine kinase inhibitor) or caffeic acid phenyl ethyl ester (Finco et al., 1994; Mahon et al., 1995; Natarajan et al., 1996), nuclear extracts from TNF-activated cells were incubated with guggulsterone and DNA-binding activity was examined. EMSA showed that guggulsterone did not modify the DNA-binding ability of NF-κB proteins prepared from cells by treatment with TNF (FIG. 3D). Therefore, guggulsterone must inhibit NF-κB activation by a different mechanism.

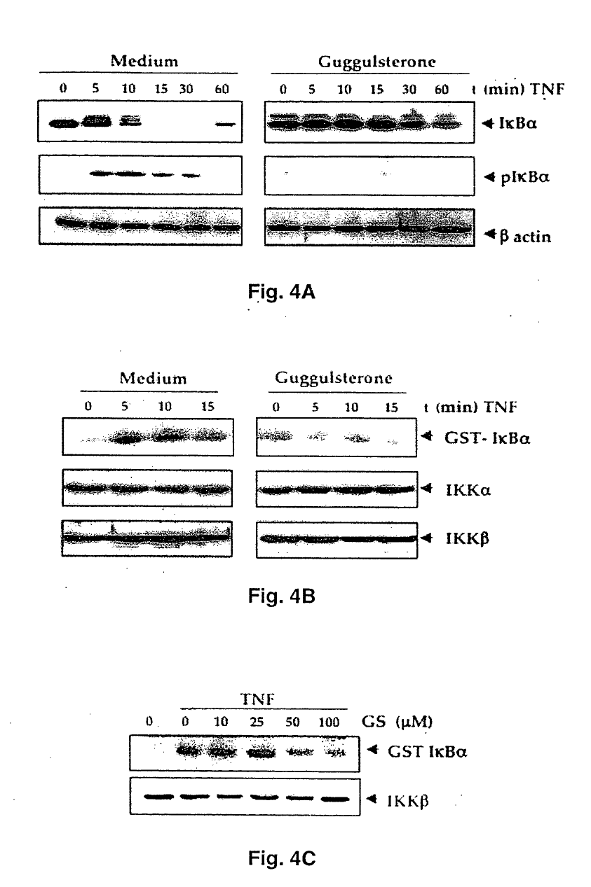

Guggulsterone Inhibits TNF-dependent IκBα Degradation and Phosphorylation: To determine whether inhibition of TNF-induced NF-κB activation was due to inhibition of IκBα degradation, normally a condition for translocation of NF-κB to the nucleus (Miyamoto et al., 1994), cells were pretreated with guggulsterone and then exposed to TNF for different times. TNF induced IκBα degradation in control cells as early as 10 min, but in guggulsterone-pretreated cells TNF had no effect on IκBα degradation (FIG. 4A, upper panel).

To determine whether guggulsterone affected TNF-induced IκBα phosphorylation, another condition for NF-κB translocation, Western blot analysis was performed using antibody that detects only the serine-phosphorylated form of IκBα. TNF induced IκBα phosphorylation as early as 5 min, and guggulsterone almost completely suppressed IκBα phosphorylation (FIG. 4A, middle panel). These results indicate that guggulsterone inhibited TNF-induced NF-κB activation through the inhibition of phosphorylation and degradation of IκBα.

Guggulsterone Inhibits TNF-induced IKK Activation: Since guggulsterone inhibits the phosphorylation of IκBα, the effect of guggulsterone on TNF-induced IKK activation, which is required for TNF-induced phosphorylation of IκBα, was tested. As shown in FIG. 4B, in an immune complex kinase assay, TNF activated IKK and the activation occurred 5 min after TNF treatment (upper panel). Guggulsterone treatment completely suppressed this activation. TNF or guggulsterone had no direct effect on the expression of either IKKα (middle panel) or IKKβ (lower panel) proteins.

Whether guggulsterone directly inhibit IKK, was assessed. When IKK was immunoprecipitated from TNF-treated cells and then incubated with different concentrations of guggulsterone, guggulsterone inhibited the phosphorylation of GST IκBα at 50 μM concentration by directly interfering with IKK activity (FIG. 4C).

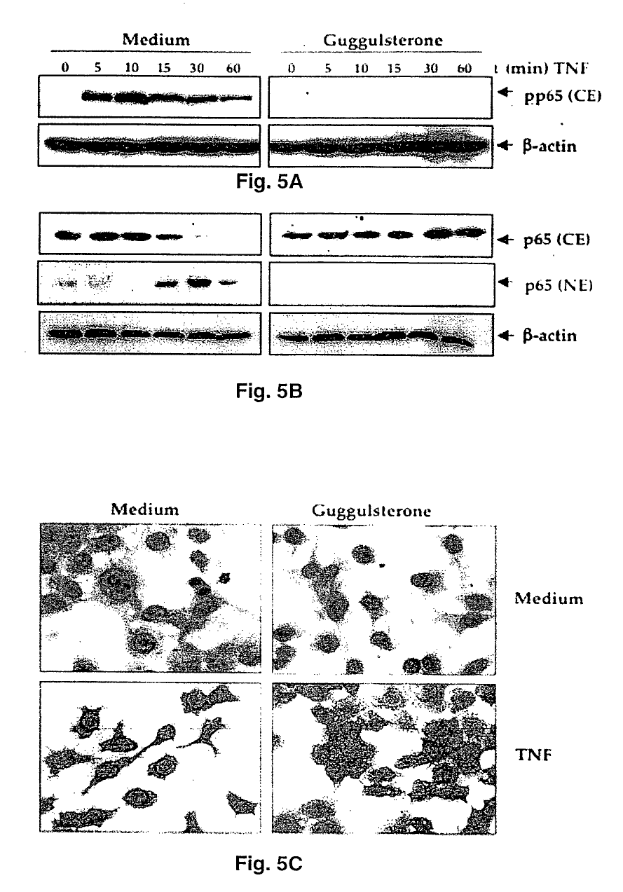

Guggulsterone Inhibits TNF-induced Phosphorylation and Nuclear Translocation of p65: The effect of guggulsterone on TNF-induced phosphorylation of p65, which is also required for transcriptional activity of p65 (Zhong et al., 1998), was assessed. As shown in FIG. 5A, TNF induced the phosphorylation of p65 in a time-dependent manner, and guggulsterone treatment suppressed p65 phosphorylation almost completely.

Whether guggulsterone affects nuclear translocation of p65, was examined by Western blot analysis. Results in FIG. 5B indicated that TNF-induced the decrease in the cytoplasmic p65 and an increase in nuclear p65. Treatment of cells with guggulsterone abolished the TNF-induced nuclear translocation of p65.

The nuclear translocation of p65 was also examined by immunocytochemistry (FIG. 5C). These results also indicated that TNF induced the nuclear translocation of p65, and guggulsterone treatment abrogated the p65 translocation.

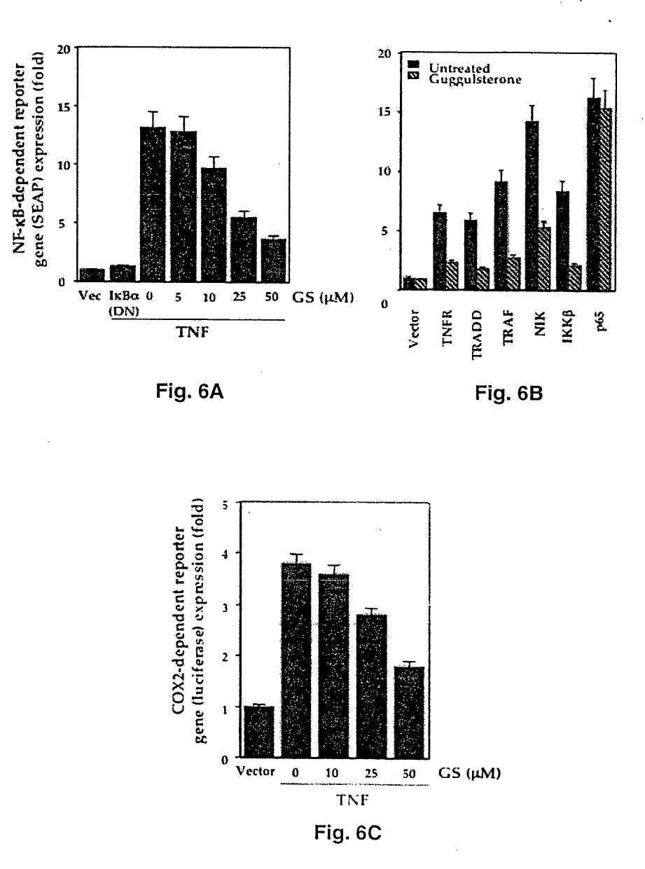

Guggulsterone Represses TNF-induced NF-κB-dependent Reporter Gene Expression: Although guggulsterone blocked NF-κB activation as shown by EMSA, DNA binding alone does not always correlate with NF-κB-dependent gene transcription, suggesting that there are additional regulatory steps (Nasuhara et al., 1999). Transient transfection of H1299 cells with the NF-κB-regulated SEAP reporter construct followed by stimulation with TNF produced an almost thirteen-fold increase in SEAP activity over vector control activity (FIG. 6A). TNF-induced SEAP activity was abolished by dominant-negative IκBα, indicating specificity. When the cells were pretreated with guggulsterone, TNF-induced NF-κB-dependent SEAP expression was inhibited by guggulsterone in a dose-dependent manner. These results demonstrate that guggulsterone inhibits NF-κB-dependent reporter gene expression induced by TNF.

TNF-induced NF-κB activation occurs through the sequential recruitment of TNFR1, TRADD, TRAF2, NIK, and IKK (Hsu et al., 1996; Simeonidis et al., 1999). To delineate the site of action of guggulsterone, cells were transfected with TNFR, TRADD, NIK, IKKβ, and p65 plasmids, and then NF-κB-dependent SEAP expression in guggulsterone-untreated and -treated cells was monitored. As shown in FIG. 6B, TNFR, TRADD, NIK, IKKβ, and p65 plasmids induced gene expression; guggulsterone suppressed reporter gene expression induced by TNFR, TRADD, NIK and IKKβ plasmids but had no effect on that induced by p65. Since IKK activation can cause the phosphorylation of IκBα and p65, it is suggested that guggulsterone inhibits NF-κB activation through inhibition of IKK.

Guggulsterone Represses TNF-induced COX2 Promoter Activity: It is well established that COX2 promoter activity is regulated by NF-κB (Yamamoto et al., 1995). The effect of guggulsterone on COX2 promoter activity was assessed. As shown in FIG. 6C, TNF activated the COX2 promoter activity and treatment of cells with guggulsterone abolished the COX2 promoter activity in a dose-dependent manner.

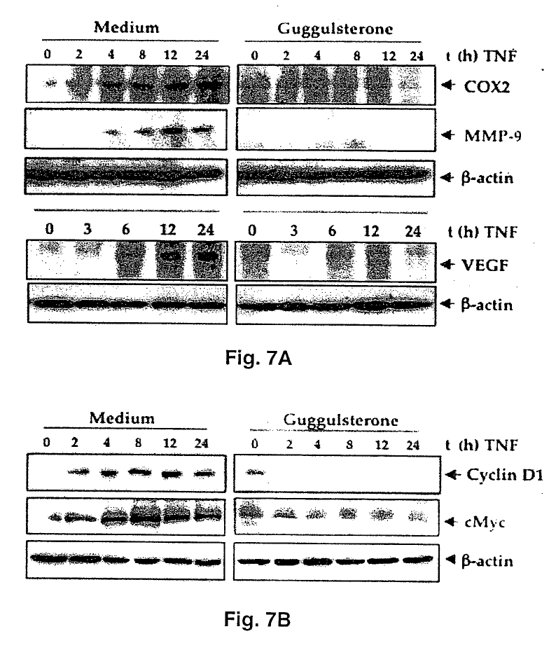

Guggulsterone Inhibits TNF-induced Gene Expression as well as TNF-induced Activation of Anti-apoptotic Gene Products: Because COX2, MMP-9, and VEGF are NF-κB regulated gene products (Yamamoto et al., 1995; Esteve et al., 2002; Huang et al., 2000), whether TNF-induced expression of these gene products is abrogated by guggulsterone was assessed. H1299 cells, either untreated or pretreated with guggulsterone, were exposed to TNF for different times. Whole-cell extracts were prepared and analyzed by Western blotting. TNF induced COX-2, MMP-9 and VEGF expression in a time-dependent manner (FIG. 7A), and guggulsterone abolished the TNF-induced expression of these gene products.

Because cyclin D1 and c-myc are NF-κB regulated gene products (Guttridge et al., 1999; Wittekindt et al., 2000), whether TNF-induced expression of these gene products is abrogated by guggulsterone was also assessed. H1299 cells, pretreated with guggulsterone, were exposed to TNF for different times. Whole-cell extracts were prepared and analyzed by Western blotting. TNF induced the expression of cell proliferative genes cyclin D1 and c-myc in a time-dependent manner (FIG. 7B), and guggulsterone abolished the expression of these gene products.

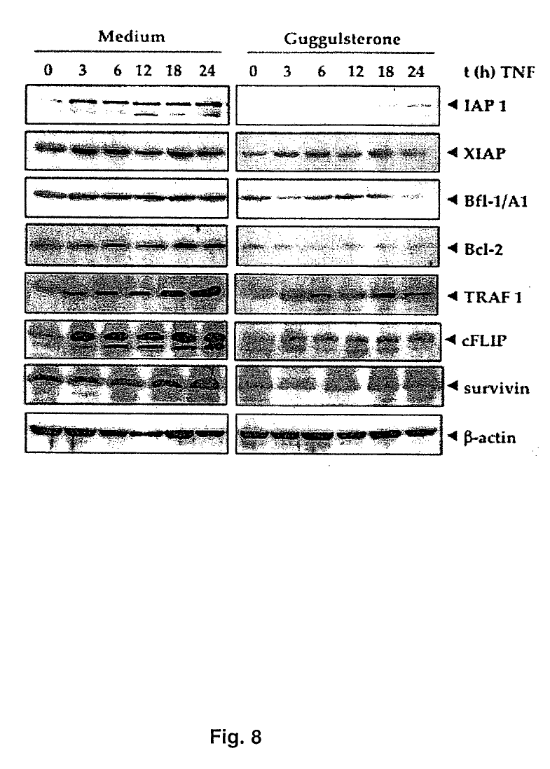

NF-κB is known to upregulate the expression of a number of genes that have been implicated in facilitating the survival of tumor cells including cIAP1, xIAP, Bfl-1, BCL-2, TRAF1, cFLIP and survivin (Zhu et al., 2001; Schwenzer et al., 1999; Chu et al., 1997; You et al., 1997; Stehlik et al., 1998; Catz et al., 2001; Grumont et al., 1999; Zong et al, 1999; Kreuz et al., 2001). The effect of guggulsterone on TNF-induced expression of cIAP1, xIAP, Bfl-1, BCL-2, TRAF1, cFLIP and surviving was assessed. The result in FIG. 8 showed that TNF induced the expression of all these proteins and this expression was inhibited by treatment of cells with gugulsterone.

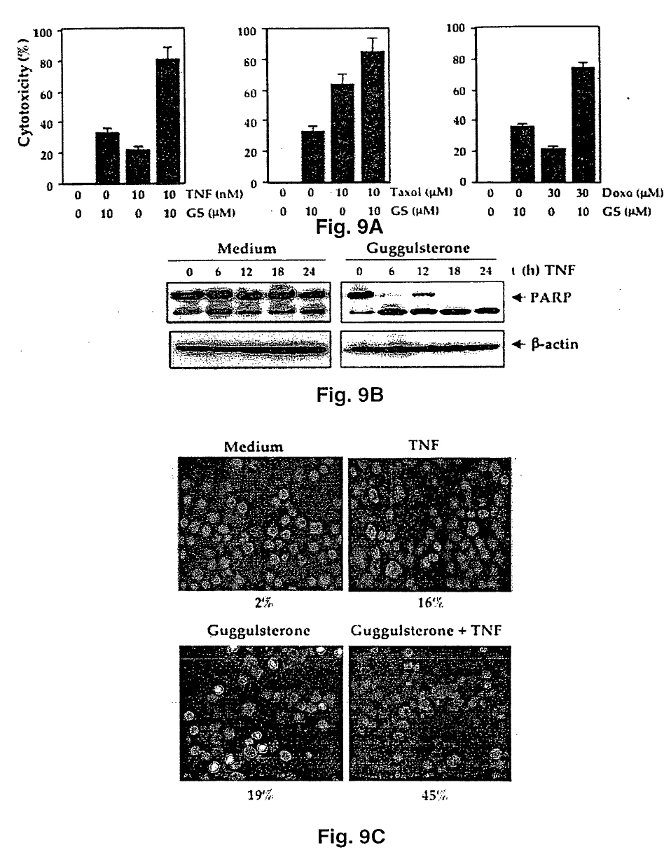

Guggulsterone Potentiates the Cytotoxic Effects of TNF and Chemotherapeutic Drugs: As NF-κB regulated products are known to suppress the apoptosis induced by TNF and chemotherapeutic agents (Van Antwerp et al., 1996; Wang et al., 1996), whether guggulsterone can enhance the apoptotic effects of TNF and the chemotherapeutic drugs, such as paclitaxel and doxorubicin, was assessed. As shown in FIG. 9A, guggulsterone enhanced the cytotoxic effects of TNF, paclitaxel and doxorubicin. Guggulsterone also enhanced the caspase-induced cleavage of PARP activated by TNF (FIG. 9B). A calcein AM-based (live/dead) cytotoxicity assay, which differentiates calcein AM (green)-stained live cells from PI stained (red) dead cells by fluorescence microscopy, showed that TNF-induced apoptosis was significantly enhanced by guggulsterone (FIG. 9C).

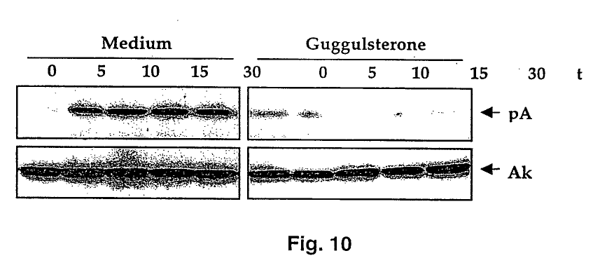

Guggulsterone Suppressed TNF-induced Activation of Akt: The results till now indicate that guggulsterone inhibits TNF-induced NF-κB activation through inhibition of IKK activation. It is possible that this inhibition is due to inhibition of an upstream kinase. Previous studies have reported that Akt can activate IKK. Thus it is possible that guggulsterone suppresses TNF-induced Akt activation. To determine the effect of guggulsterone on the activation of Akt induced by TNF, the cells were treated with guggulsterone, and then exposed to TNF (1 nM) for different times. Thereafter whole-cell extracts were prepared and Western blot analysis using phospho-specific anti-Akt antibody was performed. Results show that TNF induced Akt activation in a time-dependent manner (FIG. 10), and pretreatment with guggulsterone completely suppressed the activation. These results thus indicate that guggulsterone may inhibit IKK activation through suppression of Akt activation.

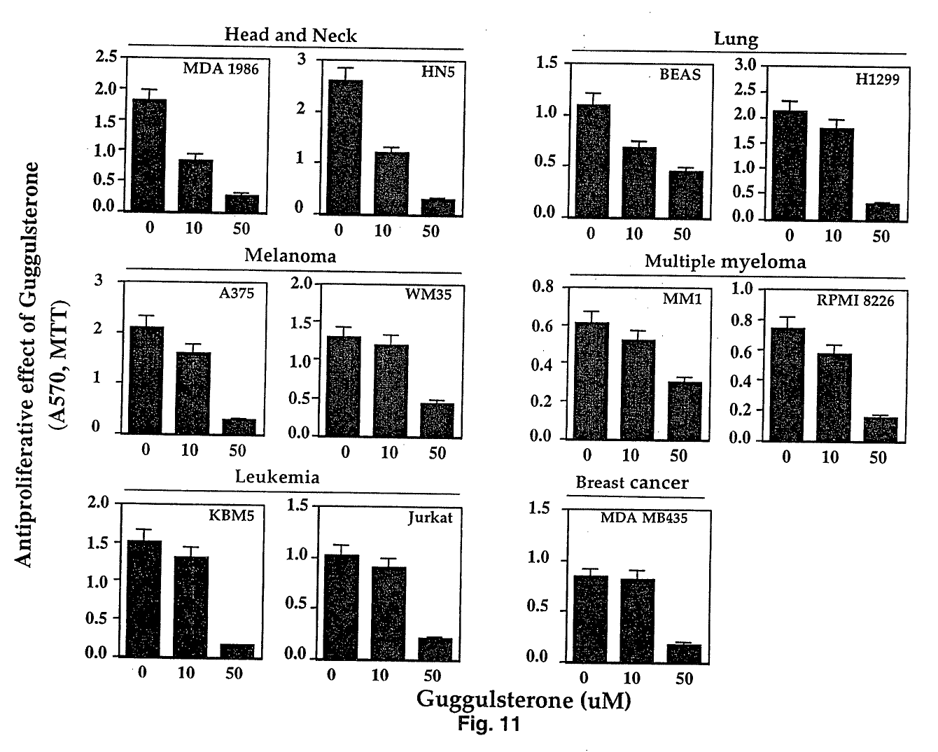

Antiproliferative Effects of Guggulsterone against Tumor Cells: Akt is a cell survival kinase. The suppression of Akt by guggulsterone suggests that it may suppress the proliferation of tumor cells. Indeed guggulsterone suppressed the proliferation of a wide variety of cells including head and neck cells, lung cells, melanoma cells, multiple myeloma cells, leukemia cells and breast cancer cells (FIG. 11).

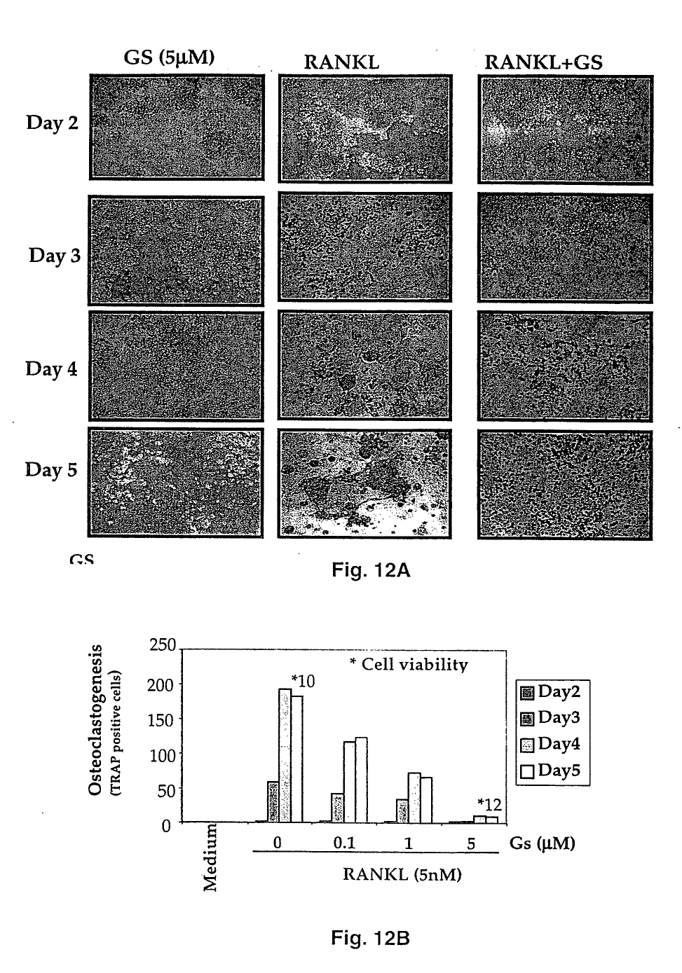

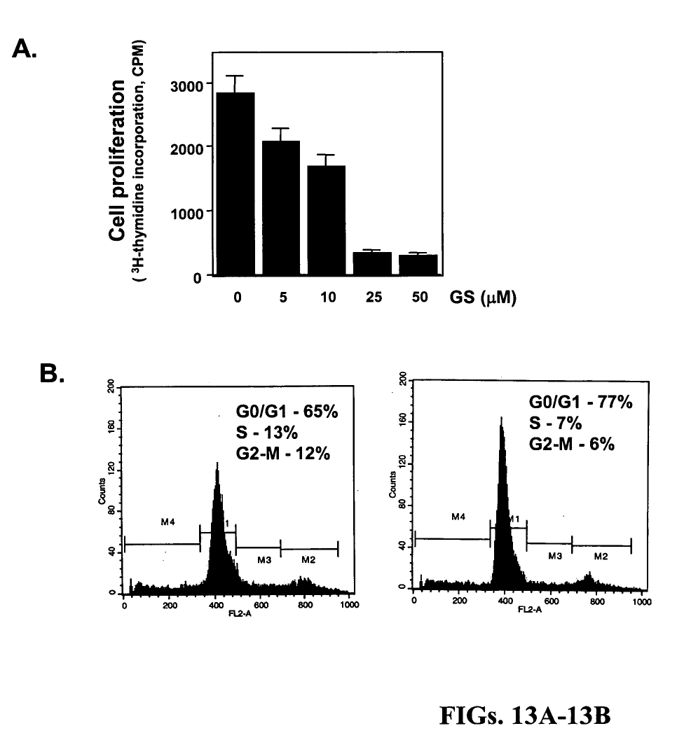

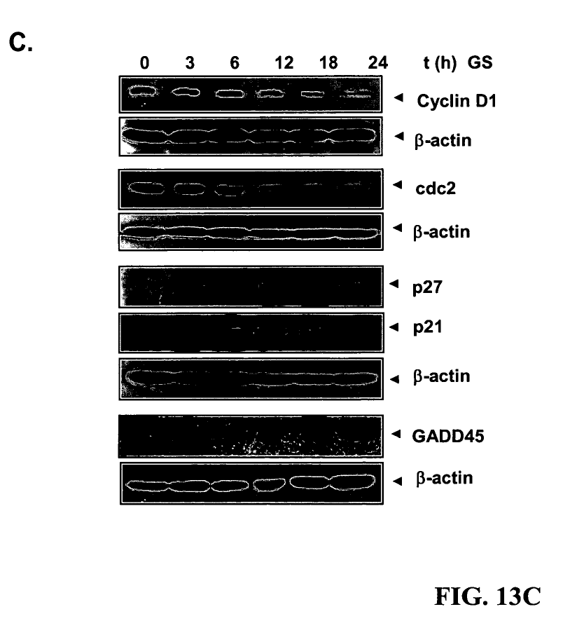

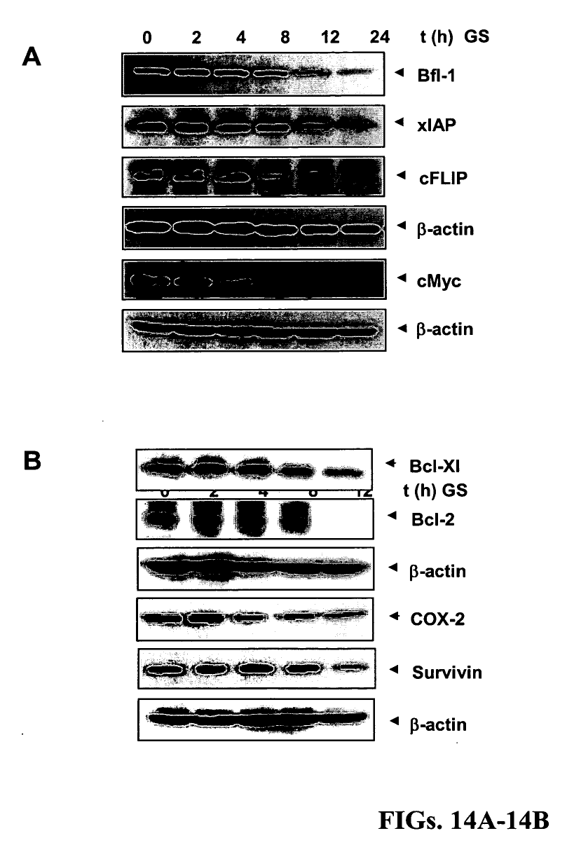

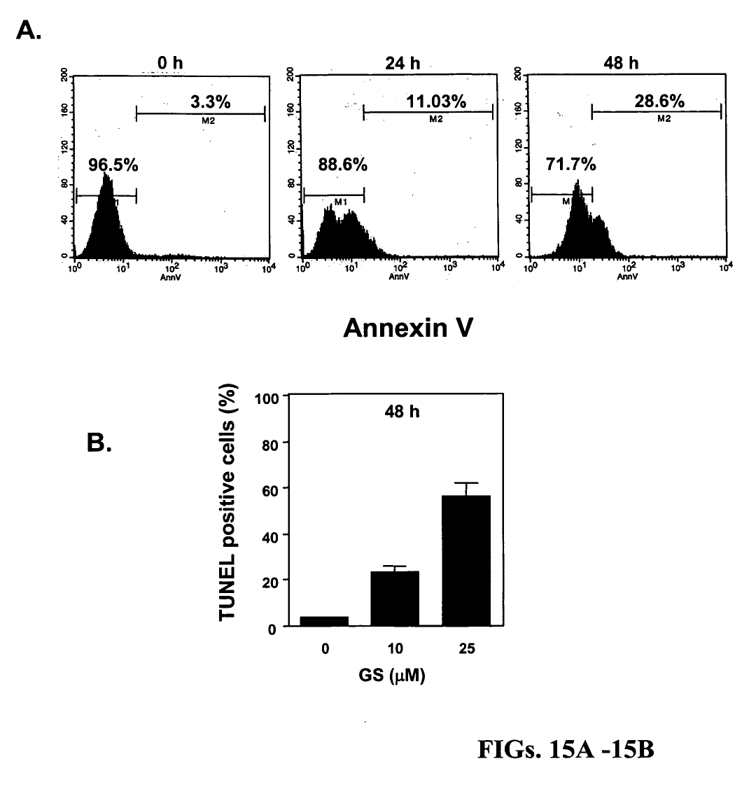

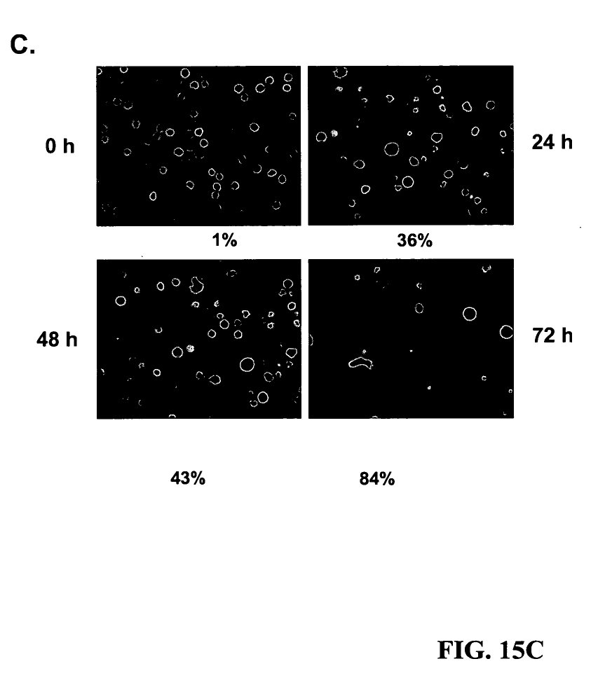

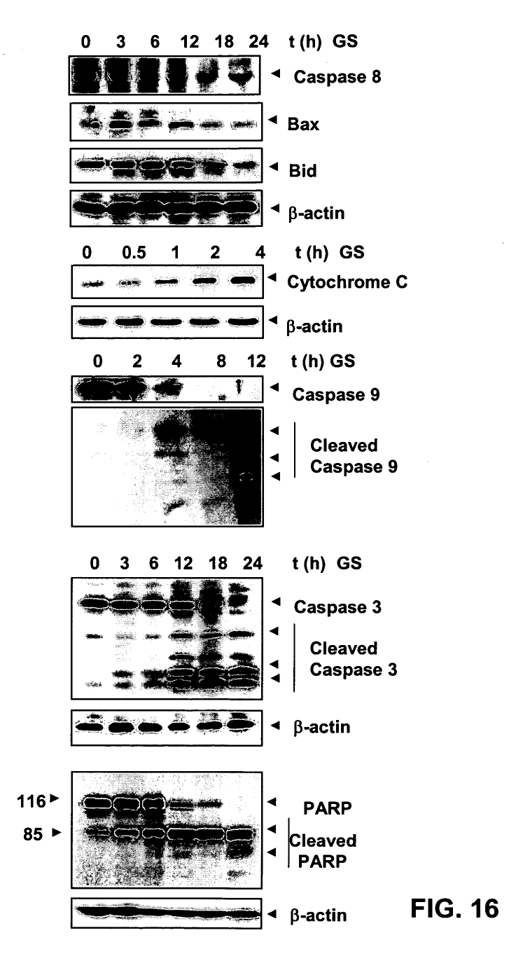

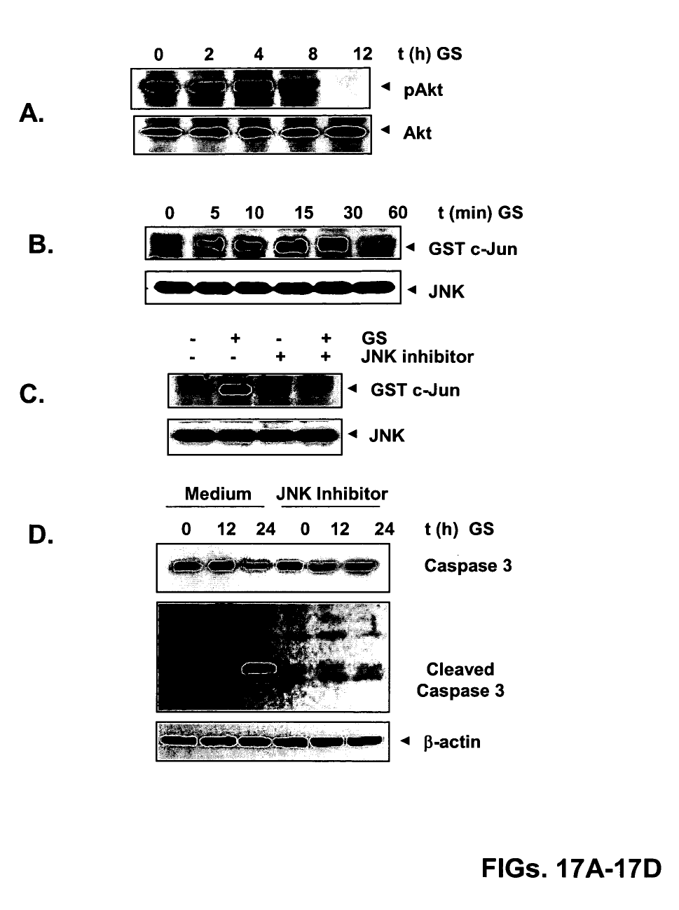

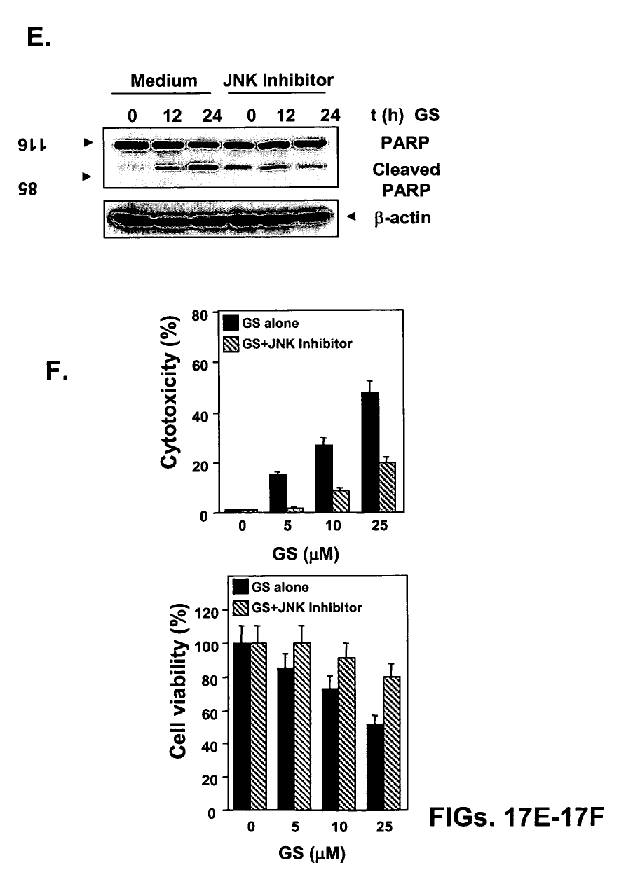

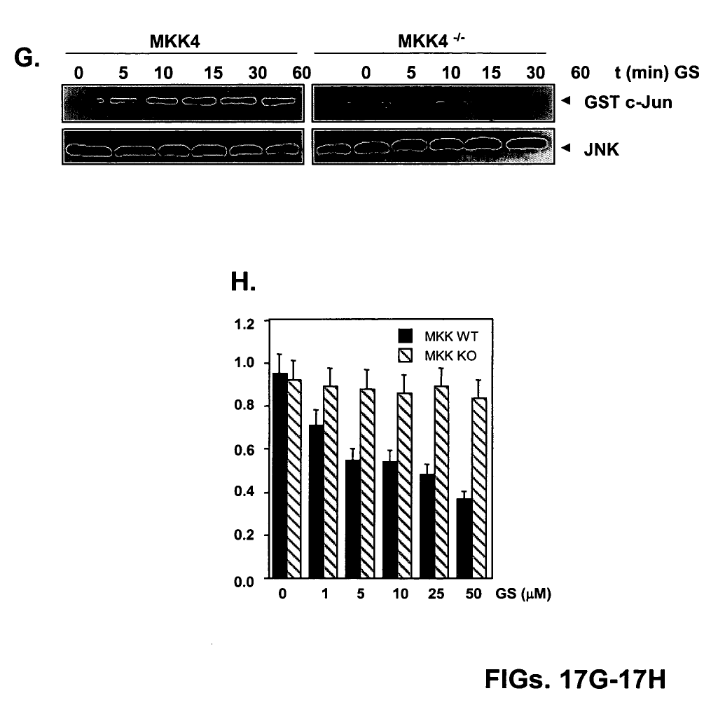

Guggulsterone inhibits RANKL-induced osteoclastogenesis: The effect of guggulsterone has been shown to suppress NF-κB activation induced by various inflammatory stimuli, inhibit the activation of IKK needed for NF-κB activation, and, found to be safe in humans, then the effect of guggulsterone on RANKL-induced NF-κB activation and on osteoclastogenesis in osteoclast precursor cells was assessed. The result demonstrates that RANKL induces NF-κB activation through activation of IκB kinase (IKK), and IκBα, phosphorylation and degradation and guggulsterone inhibits RANKL-induced NF-κB activation and osteoclastogenesis (FIGS. 12A and 12B).

### Example 2

### Guggulsterone Inhibits Proliferation of Various Human Tumor Cell Lines

A. Material and Methods

Materials. Z-Guggulsterone, obtained from Steraloids, Inc. (Newport, R.I.), was dissolved in dimethyl sulfoxide (DMSO) as a 10 mM stock solution and stored at −20° C. Penicillin, streptomycin, RPMI 1640 medium, FBS and lipofectamine 2000 were obtained from Invitrogen (Grand Island, N.Y.). The following polyclonal antibodies were obtained from Santa Cruz Biotechnology, Inc. (Santa Cruz, Calif.): anti-p65, against the epitope corresponding to amino acids mapping within the amino terminal domain of human NF-κB p65; anti-p50, against a peptide 15 amino acids long mapping at the nuclear localization sequence region of NF-κB p50; anti-IκBα, against amino acids 297-317 mapping at the carboxy terminus of IκBα/MAD-3; anti-c-Rel, anti-cyclin D1 against amino acids 1-295, which represents full-length cyclin D1 of human origin; anti-MMP-9, anti-polyadenosine ribose polymerase (PARP); anti-IAP1; anti-IAP2; anti-Bcl-2; anti-Bfl-1/A1; and GADD45β. Phospho-IκBα (Ser32) antibody was purchased from New England Bio Labs (Beverly, Mass.). Anti-COX-2, and anti-XIAP antibodies were obtained from BD Biosciences (San Diego, Calif.). Anti-IKKα and anti-IKKβ antibodies were kindly provided by Imgenex (San Diego, Calif.).

Cell lines. The cell lines used included chronic myelogenous leukemia (KBM-5), human monocytic leukemia (U937), human melanoma (A375, WM35), human lymphoblastic leukemia (Jurkat), human chronic myelogenous leukemia (K562), human non-small cell lung carcinoma (H1299), human Norman bronchial epithelial cells (BEAS-2B), human multiple myeloma (U266, MM1), human head and neck cancer (HN5, SCC4, FADU), human breast cancer (MCF-7) and human ovarian cancer (HEY8, SKOV3). All cell lines were obtained from the American Type Culture Collection (Manassas, Va.). KBM-5 cells were cultured in Iscove's modified DMEM with 15% FBS, BEAS-2B cells were cultured in keratinocyte serum-free media, Melanoma cell lines were cultured in RPMI 1640 supplemented with 10% HEPES and 10% FBS and all other cell lines were cultured in RPMI 1640 medium with 10% FBS. Media were supplemented with 100 U/ml penicillin and 100 μg/ml streptomycin. Media were supplemented with 100 U/ml penicillin, and 100 μg/ml streptomycin.

Cytotoxicity assay. The cytotoxic effects of guggulsterone was determined by the MTT uptake method. Briefly, 5000 cells were incubated with guggulsterone in triplicate in a 96-well plate at 37° C. MTT solution was then added to each well. After a 2-h incubation at 37° C., extraction buffer (20% SDS, 50% dimethylformamide) was added, the cells were incubated overnight at 37° C., and the OD was then measured at 570 nm using a 96-well multiscanner (Dynex Technologies, MRX Revelation, Chantilly, Va.).

Thymidine incorporation assay. The antiproliferative effects of guggulsterone were also monitored by the thymidine incorporation method. For this, 5000 cells in 100 μL medium were cultured in triplicate in 96-well plates in the presence or absence of guggulsterone for 72 hours. At 6 hours before the completion of experiment, cells were pulsed with 0.5 μCi (0.0185 MBq) ³H-thymidine, and the uptake of ³H-thymidine was monitored by means of a Matrix-9600 β-counter (Packard Instruments, Downers Grove, Ill.).

Flow cytometric analysis. To determine the effect of guggulsterone on the cell cycle, U937 cells were treated for different times, washed, and fixed with 70% ethanol. After an overnight incubation at −20° C., cells were washed with PBS, and then suspended in staining buffer (Propidium iodide, 10 μg/ml; Tween-20, 0.5%; RNase, 0.1% in PBS). The cells were analyzed using a FACS Vantage flow cytometer that uses CellQuest acquisition and analysis programs (Becton Dickinson, San Jose, Calif.). Gating was set to exclude cell debris, cell doublets, and cell clumps.

Western blot analysis. Thirty to fifty micrograms of cytoplasmic, nuclear or whole cell protein was resolved on 10% SDS-PAGE gel, transferred to a nitrocellulose membrane, blocked with 5% non-fat milk, and probed with specific antibodies as per manufacturer's recommended protocol. The blots were washed, exposed to HRP-conjugated secondary antibodies for 1 h, and detected by ECL reagent (Amersham Pharmacia Biotechnology, Piscataway, N.J.). The bands were quantitated using a Personal Densitometer Scan v1.30 using Imagequant software version 3.3 (Molecular Dynamics).

Annexin V Assay. One of the early indicators of apoptosis is the rapid translocation and accumulation of the membrane phospholipid phosphatidylserine from the cell's cytoplasmic interface to the extracellular surface. This loss of membrane asymmetry can be detected using the binding properties of annexin V. To detect apoptosis, annexin V antibody conjugated with the fluorescent dye FITC was used. Briefly, 1×10⁶ cells were pretreated with 10 μM guggulsterone for various time points and then subjected to annexin V staining. Cells were washed, stained with FITC-conjugated anti-annexin V antibody, and then analyzed with a flow cytometer (FACSCalibur; BD Biosciences).

TUNEL Assay. Apoptosis was also assessed by the terminal deoxynucleotidyl transferase-mediated deoxyuridine triphosphate nick end-labeling (TUNEL) method, which examines DNA strand breaks that occur during apoptosis, using an in situ cell death detection reagent (Roche Molecular Biochemicals, Mannheim, Germany). Assays were performed using 5×10⁵ cells that were incubated with 10 μM guggulsterone for 24 h and then fixed with 4% paraformaldehyde, and permeabilized with 0.1% Triton-X 100 in 0.1% sodium citrate. After being washed, the cells were incubated with reaction mixture for 60 min at 37° C. Stained cells were analyzed with a flow cytometer (FACSCalibur).

Live and dead assay. To measure apoptosis, the Live and Dead assay (Molecular Probes) was used, which determines intracellular esterase activity and plasma membrane integrity. This assay employs calcein, a polyanionic dye, which is retained within the live cells and provides green fluorescence. It also employs the ethidium monomer dye (red fluorescence), which can enter the cells only through damaged membranes and bind to nucleic acids but is excluded by the intact plasma membrane of live cells. Briefly, 1×10⁵ cells are incubated with 10 μM guggulsterone for various time points at 37° C. Cells are stained with the Live and Dead reagent (5 μM ethidium homodimer, 5 μM calcein-AM) and then incubated at 37° C. for 30 min. Cells were analyzed under a fluorescence microscope (Labophot-2).

JNK kinase assay. To determine the effect of guggulsterone on the kinase activity of JNK, JNK complex from whole-cell extracts was precipitated with antibody against JNK1, followed by treatment with protein A/G-Sepharose beads (Pierce, Rockford, Ill.). After 2 h of incubation, the beads were washed with lysis buffer and then assayed in kinase assay mixture containing 50 mM HEPES (pH 7.4), 20 mM MgCl₂, 2 mM dithiothreitol, 20 μCi of [γ-³²P]ATP, 10 μM unlabeled ATP, and 2 μg of substrate GST-c-Jun (1-79). The immunocomplex was incubated at 30° C. for 30 min and then boiled with SDS sample buffer for 5 min. Finally, the protein was resolved on 10% SDS-PAGE, the gel was dried, and the radioactive bands were visualized using the PhosphorImager. To determine the total amount of JNK1 in each sample, whole-cell extracts were subjected to Western blot analysis using anti-JNK1 antibody.

Measurement of cytochrome c release. To determine the effect of guggulsterone on cytochrome c release, cells were treated with guggulsterone as indicated and then the cytosolic extracts were prepared as described (Yang, 1997). Briefly, the cells were washed with PBS, resuspended in the buffer containing 0.25 M sucrose, 30 mM Tris-HCl (pH 7.9), 1 mM EDTA, 1 mM PMSF, 2 mM sodium orthovanadate, 10 mM NaF, 2 μg/ml leupeptin, and 2 μg/ml aprotinin and then homogenized gently with a glass Dounce homogenizer for 20 strokes. The homogenates were centrifuged at 2000 rpm for 10 min to remove nuclei, and the supernatants were centrifuged at 14,000 rpm for 30 min to remove mitochondria and other insoluble fragments. The supernatants were again centrifuged as above to ensure complete removal of mitochondria. Protein (50 μM) was subjected to 15% SDS-PAGE, and then Western blot analysis was performed using anti-cytochrome c antibody.

PARP cleavage assay. For detection of cleavage products of PARP, whole-cell extracts were prepared by subjecting guggulsterone-treated cells to lysis in lysis buffer (20 mM Tris, pH 7.4; 250 mM NaCl; 2 mM EDTA, pH 8.0; 0.1% Triton-X 100; 0.01 μg/ml aprotinin; 0.005 μg/ml leupeptin; 0.4 mM PMSF; and 4 mM NaVO₄). Lysates were spun at 14000 rpm for 10 min to remove insoluble material, resolved by 10% SDS PAGE, and probed with PARP antibodies.

B. Results

The antiinflammatory effect of guggulsterone is well described, whether this steroid has any role in cancer is not yet understood. In the present study, it is shown that guggul inhibits the proliferation of human leukemia, human head and neck carcinoma, human multiple myeloma, human lung carcinoma, human melanoma, human breast carcinoma, and human ovarian cancer cell lines. Guggulsterone also inhibited the proliferation of drug resistant cancer cells (e.g., STI-resistant K562 cells, dexamethasone resistant MM1 cells and doxorubicin resistant breast cancer cell lines). When examined it was found that guggulsterone modulated the cytostatic pathway, apoptotic pathway, and JNK pathway. Guggulsterone arrested the cell cycle in S-phase and this correlated with a decrease in the levels of cyclin D1 and cdc2 and a concomitant increase in the levels of cyclin dependent kinase inhibitor p21, p27 and the growth arrest and DNA-damage-inducible protein GADD45β. Guggulsterone treatment increased the number of Annexin V and TUNEL positive cells. Guggulsterone inhibited the gene products involved in antiapoptosis (xIAP, Bfl-1/A1, Bcl-2, BclX1, cFLIP, and survivin), proliferation (c-myc) and metastasis (COX2). Guggulsterone induced both receptor-mediated apoptosis through the activation of caspase 8 and non-receptor mediated apoptosis through the mitochondrial cytochrome c release. This led to the activation of caspase 3 and PARP cleavage. Guggulsterone mediated apoptosis was mediated through the suppression of Akt-activation and activation of JNK. Overall, results indicate that guggulsterone can inhibit cell proliferation and induce apoptosis through the activaton of JNK, suppression of Akt, and downregulation of anti-apoptotic gene expression.

### Example 3

### Guggulsterone Suppresses Osteoclastogenesis

A. Material and Methods

Materials. The rabbit polyclonal antibodies (Abs) to IκBα, p50, p65, were purchased from Santa Cruz Biotechnology (Santa Cruz, Calif.). Ab against phospho-IκBα was purchased from Cell Signaling Technology (Beverly, Mass.). Anti-IKK-α and anti-IKK-β Abs were kindly provided by Imgenex (San Diego, Calif.). Goat anti-rabbit HRP conjugate was purchased from Bio-Rad (Hercules, Calif.), goat anti-mouse HRP and BioCoat Osteologic Bone Cell Culture System from BD Biosciences (San Jose, Calif.), and MTT from Sigma-Aldrich (St. Louis, Mo.). GS with a purity 98% was purchased from LKT Laboratories (St. Paul, Minn.) and was prepared as a 20 mM solution in DMSO and then further diluted in cell culture medium. DMEMF-12, FBS, 0.4% trypan blue vital stain, and antibiotic-antimycotic mixture were obtained from Invitrogen (Carlsbad, Calif.). Protein A/G-Sepharose beads were obtained from Pierce (Rockford, Ill.). [γ-³²P]ATP was from ICN Pharmaceuticals (Costa Mesa, Calif.). Highly purified recombinant murine TNF-α was provided by Genentech (South San Francisco, Calif.).

Cell lines. The mouse macrophage cell line RAW 264.7 was obtained from American Type Culture Collection (Manassas, Va.). RAW 264.7 cells were cultured in DMEM-F12 medium supplemented with 10% FBS and antibiotics. This cell line has been shown to express RANK and differentiate into tartrate resistance acid phosphatase (TRAP)-positive, functional osteoclasts when co-cultured with soluble RANKL (Hsu et al., 1999). Moreover, RANKL has been shown to activate NF-κB in these cells (Wei et al., 2001). TRAP staining was performed using a leukocyte acid phosphatase kit (387-A) from Sigma-Aldrich. MDA-MB-468 (human breast adenocarcinoma) cells were obtained from American Type Culture Collection. This cells were cultured in MEM containing 10% FBS, 100 μM nonessential amino acids, 1 mM pyruvate, 6 mM L-glutamine. U266 cells were cultured in RPMI 1640 medium with 10% FBS, and Culture media were also supplemented with 100 U/ml penicillin and 100 μg/ml streptomycin.

Osteoclast differentiation assay. RAW 234.7 cells were cultured in 24-well dishes at a density of 1×10⁴ cells per well and were allowed to adhere overnight. Medium was then replaced and the cells were treated with 5 nM (100 ng/ml) RANKL. At day 5, cultures were stained for TRAP expression as described (Shevde et al., 2000) using an acid phosphatase kit, and the total number of TRAP-positive multinucleated osteoclasts (3 nuclei) per well were counted.

Electrophoretic mobility shift assays (EMSA). To determine NF-κB activation, EMSA was performed as described previously (Chaturvedi et al., 1994). Briefly, nuclear extracts prepared from TNF-treated cells were incubated with ³²P-end-labeled 45-mer double-stranded NF-κB oligonucleotide (15 μg of protein with 16 fmol of DNA) from the human immunodeficiency virus long terminal repeat, 5′-TTGTTACAAGGGACTTTCCGCTGGGGACTTTCCAGGG AGGCGTGG-3′ (SEQ ID NO:5)(boldface indicates NF-κB binding sites) for 30 min at 37° C., and the DNA-protein complex formed was separated from free oligonucleotide on 6.6% native polyacrylamide gels. A double-stranded mutated oligonucleotide, 5′-TTGTTACAACTCAC TTTCCGCTGCTCACTTTCCAGGGAGGCGTGG-3′ (SEQ ID NO:6), was used to examine the specificity of binding of NF-κB to the DNA. The specificity of binding was also examined by competition with the unlabeled oligonucleotide. For supershift assays, nuclear extracts prepared from TNF-treated cells were incubated with antibodies against either p50 or p65 of NF-κB for 15 min at 37° C. before the complex was analyzed by EMSA. The dried gels were visualized, and radioactive bands quantitated by a PhosphorImager (Molecular Dynamics, Sunnyvale, Calif.) using Imagequant software.

Western blot analysis. To determine the levels of protein expression in the cytoplasm or nucleus, extracts were prepared (Majumder and Aggarwal, 2001) and fractionated by SDS-polyacrylamide gel electrophoresis (PAGE). After electrophoresis, the proteins were electrotransferred to nitrocellulose membranes, blotted with each antibody, and detected by ECL regent (Amersham, Piscataway, N.J.). The bands obtained were quantitated using NIH imaging software (NIH, Bethesda, Md.).

IKK assay. To determine the effect of GS on TNF-induced IKK activation, IKK assay was performed by a method described previously (Manna et al., 2000). To determine the total amounts of IKK-α and IKK-β in each sample, 400 μg of the whole-cell protein was resolved on 7.5% SDS-PAGE, electrotransferred to a nitrocellulose membrane, and then blotted with either anti-IKK-α or anti-IKK-β antibodies.

B. Results

The effect of GS on RANKL-induced NF-κB activation and on osteoclastogenesis in the murine monocytic cell line RAW 264.7 were investigated.

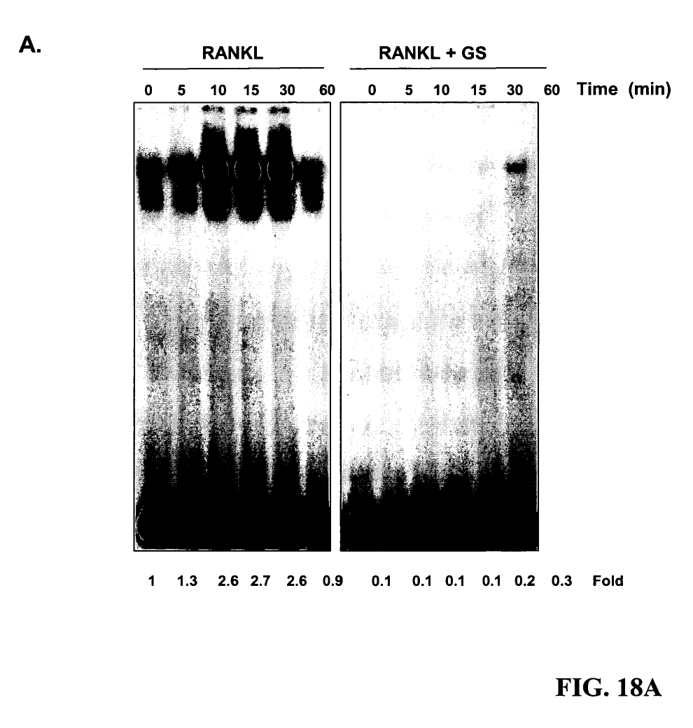

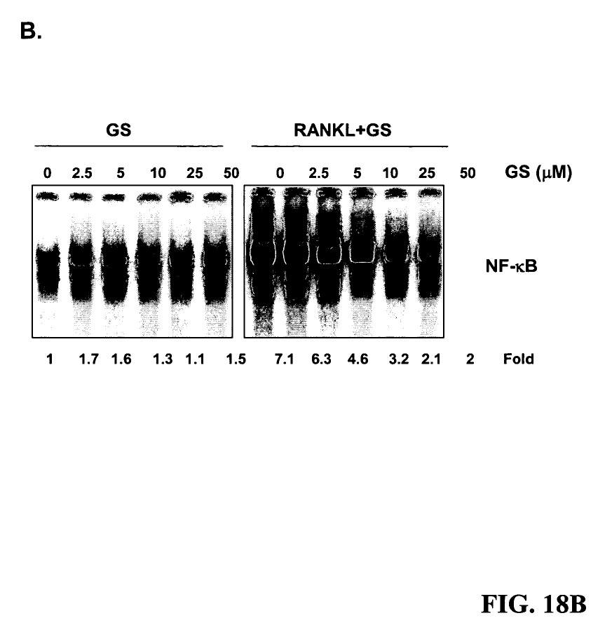

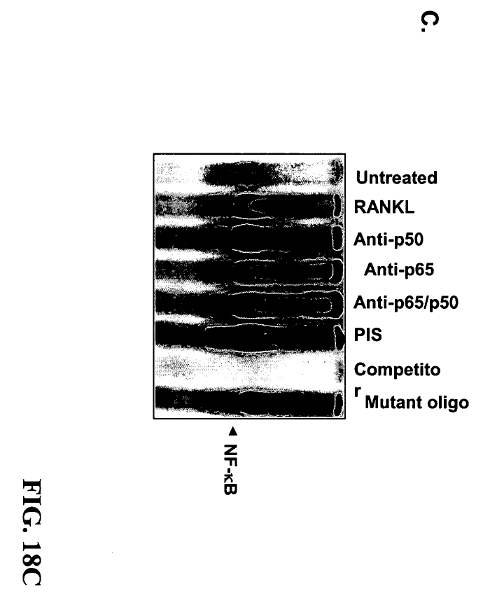

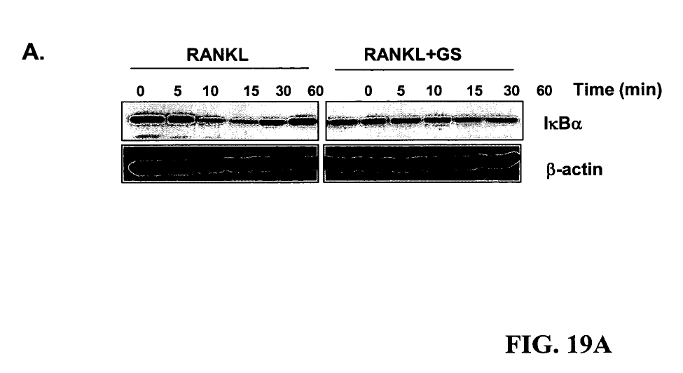

GS inhibits RANKL-induced NF-κB activation. To determine the effect of GS on RANKL-induced NF-κB activation in RAW 264.7 cells, cells were coincubated with GS and RANKL, prepared nuclear extracts, and assayed NF-κB activation by EMSA. RANKL activated NF-κB maximally within 30 min, and GS completely abrogated the RANKL-induced NF-κB activation (FIG. 18A). The inhibition of NF-κB by GS increased with dose. Complete inhibition was observed at a 50 μM concentration of GS (FIG. 18B). Supershift assay of NF-κB-DNA probe binding showed that RANKL-activated NF-κB consisted of p65 and p50 subunits (FIG. 18C). Reaction mixtures containing Abs to p50 or p65 showed either lesser NF-κB-DNA complex (with anti-p50) or a further shift in the NF-κB-DNA complex band (with anti-p65). The specificity of the RANKL-induced NF-κB-DNA complex was further confirmed by demonstrating that the binding was and was abolished by the presence of a 100-fold excess of unlabeled κB-oligonucleotides (FIG. 18C).

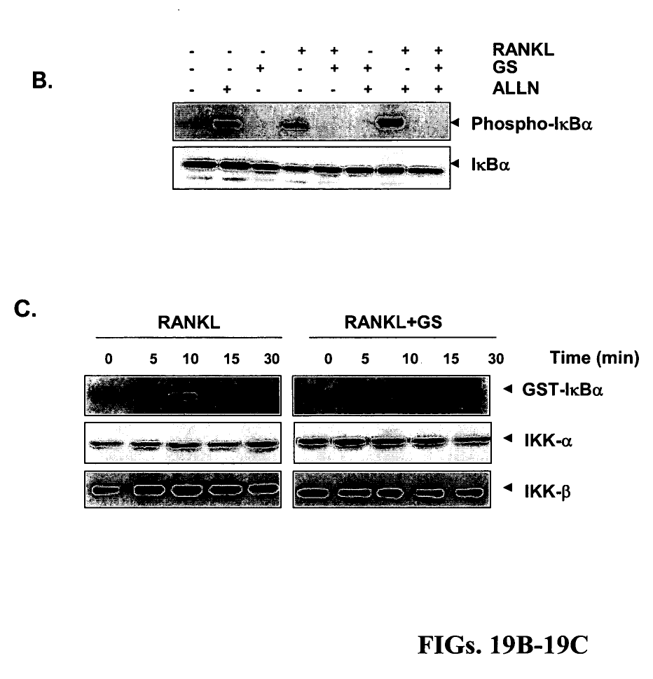

GS inhibits RANKL-induced IκBα phosphorylation and degradation through inhibition of IKK activity. Activation of NF-κB by most agents requires phosphorylation and degradation of its inhibitory subunit IκBα. To investigate the mechanism involved in the inhibition of NF-κB activation by GS, the effects of GS treatment on the levels of IκBα were assessed by Western blot analysis. The IκBα level dropped within 15 min in the cells treated with RANKL, and returned to normal levels within 60 min of treatment (FIG. 19A, left). In contrast, cells pretreated with GS suppressed RANKL-induced IκBα degradation (FIG. 19A, right). Next the effect of GS on the RANKL-induced phosphorylation of IκBα was assessed, which occurs before its dissociation, ubiquitination, and degradation (Rothwarf and Karin, 1999). Western blot analysis for phospho-IκBα in FIG. 19B clearly indicates that RANKL induced IκBα phosphorylation in RAW 264.7 cells and that GS eliminated the RANKL-induced phosphorylation. Treatment of cells with GS alone did not result in phosphorylation of IκBα. It is noticeable that the content of IκBα in GS-treated sample was lesser than the control; quantitating of IκBα to β-actin ratio indicates that GS treatment down-regulates the expression of IκBα and inhibits the RANKL-induced degradation of IκBα. Because IKK phosphorylates IκBα (DiDonato et al., 1997), whether GS alters the activity or the levels of IKK were studied. In in vitro IKK assay, cells treated with RANKL showed a sharp rise in IKK activity as indicated by the phosphorylation of IκBα within 10 min. In contrast, cells pretreated with GS could not phosphorylate GST-IκBα upon RANKL treatment (FIG. 19C). To check whether the apparent loss of IKK activity was due to the loss of IKK protein expression, the expression levels of the IKK subunits IKK-α and IKK-β were tested by Western blot analysis. Results in FIG. 19C clearly showed that GS treatment did not alter the expression of IKK-α and IKK-β.

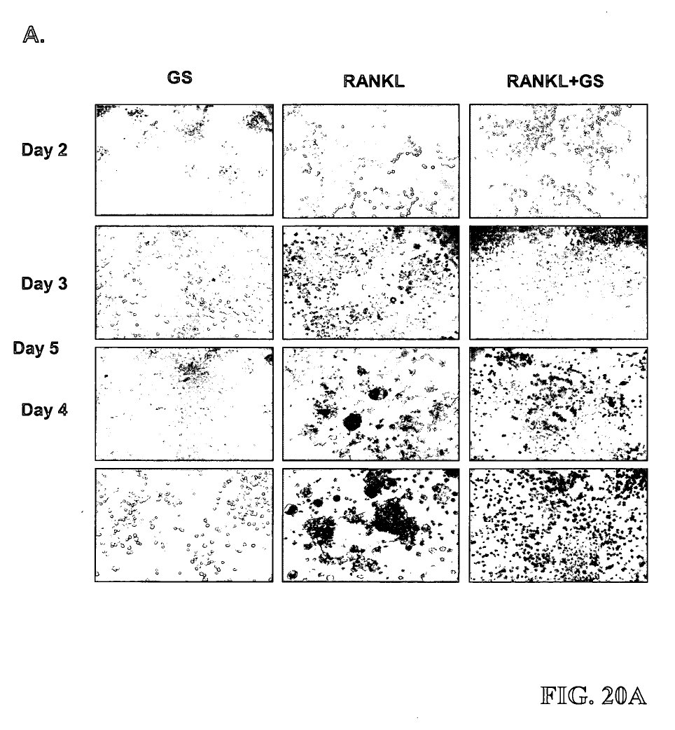

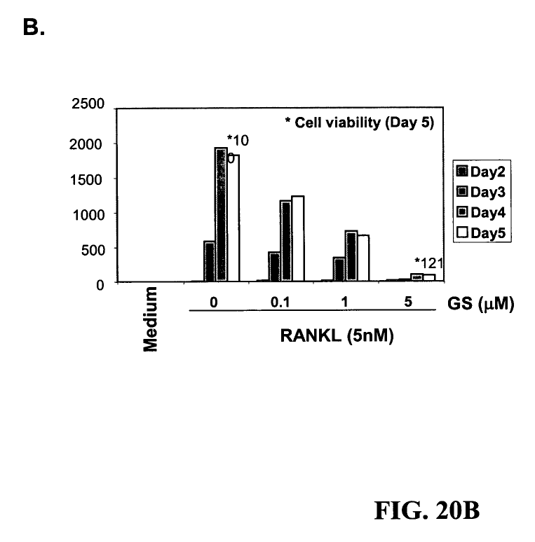

GS inhibits RANKL-induced osteoclastogenesis in RAW 264.7 cells. Next the effect of GS on osteoclastogenesis was assessed. RAW 264.7 cells were incubated with different concentrations of GS in the presence of RANKL and allowed to grow and differentiate into osteoclasts. FIG. 20A illustrates that RANKL induced osteoclasts both in the presence and absence of GS. However, the number of osteoclasts decreased with increasing concentration of GS (FIG. 20B).

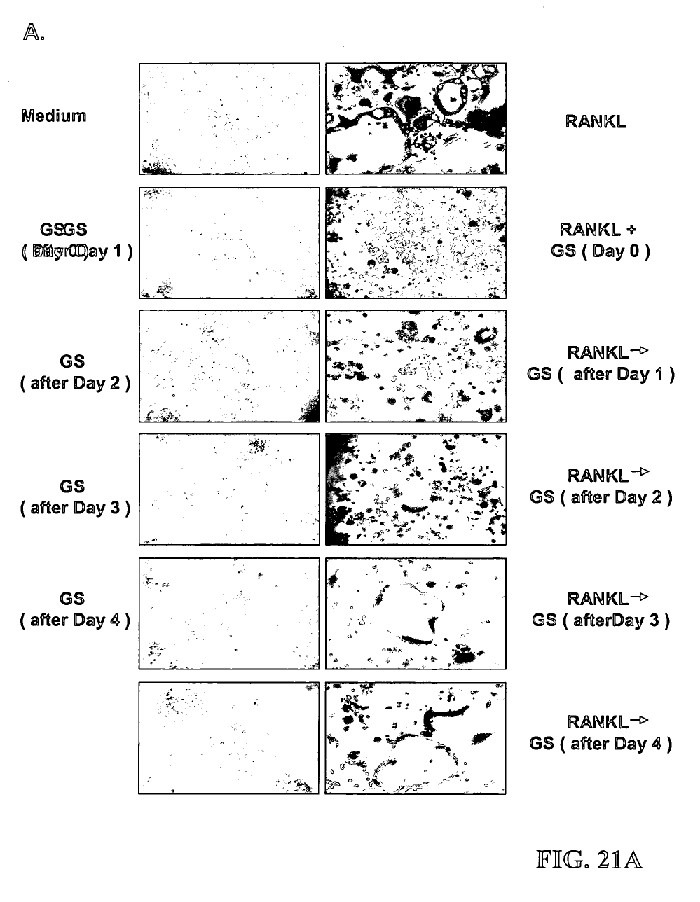

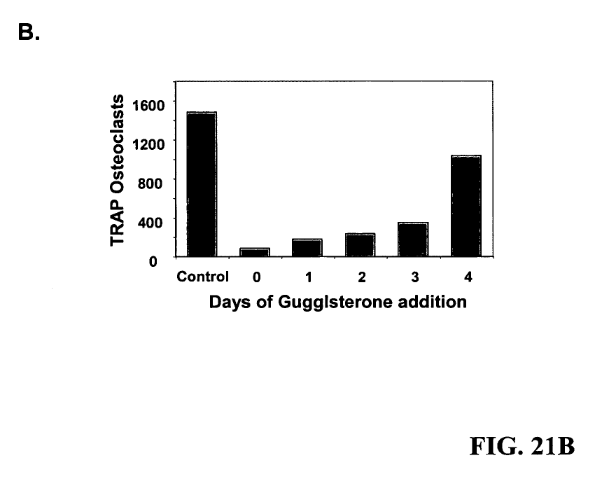

GS acts early in the pathway leading to RANKL-induced osteoclastogenesis. It normally takes up to 5 days for RAW 264.7 cells to differentiate into osteoclasts in response to RANKL. To determine how early in this pathway GS acts, the RAW 264.7 cells were treated with RANKL, added GS on different days, and then checked its effect on osteoclast formation. GS inhibited osteoclastogenesis even when the cells were exposed 24 h after the RANKL treatment (FIG. 21A). However the inhibitory effect decreased significantly when cells were treated with GS 3 days after RANKL treatment (FIG. 21B).

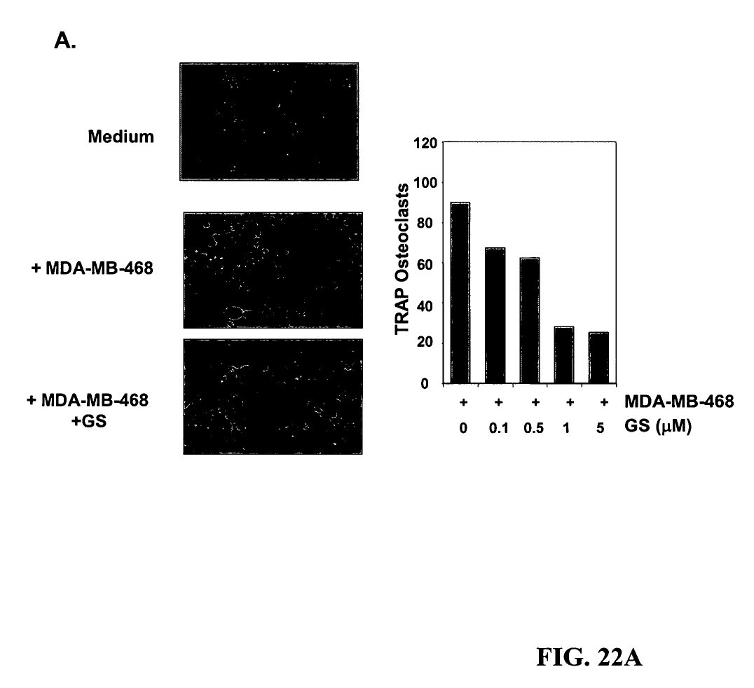

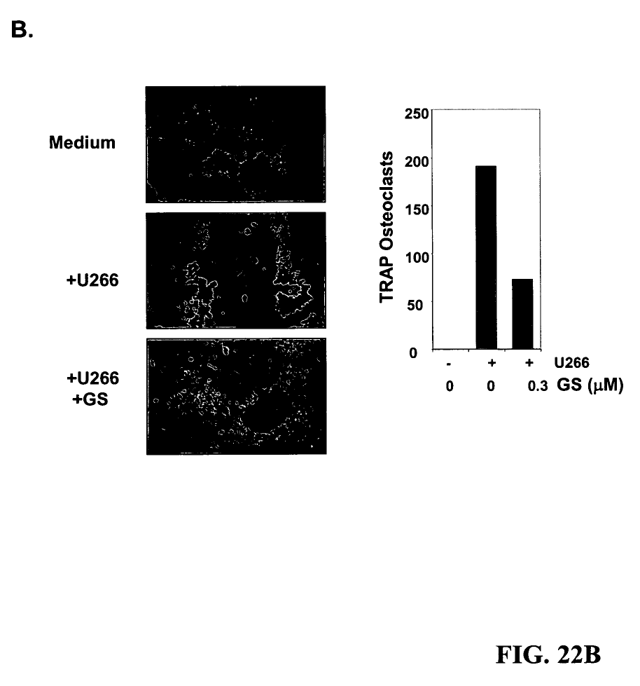

Activation of NF-κB is critical for coincubation-induced osteoclastogenesis. Osteoclastogenesis is commonly associated with breast cancer and others such as multiple myeloma and is mediated through RANKL (Dai et al., 2004). The co-incubation of breast cancer MDA-MB-468 cells-induced and U266 cells-induced osteoclast differentiation with GS was found to suppressed osteoclastogenesis (FIGS. 22A and 22B). Also, it was found that co-incubation of multiple myeloma U266 cells with macrophages induced osteoclast differentiation was suppressed (FIGS. 22C and 22D).

### Example 4

### Gulggulsterone Inhibits Constitutive and Interleukin-6-Inducible STAT3 Phosphorylation in Human Multiple Myeloma Cells

A. Materials and Methods

Materials. Human MM cell lines U266, RPMI 8226, and MM.1S were obtained from the American Type Culture Collection (Rockville, Md.). Cell lines U266 (ATCC#TIB-196) and RPMI 8226 (ATCC#CCL-155) are plasmacytomas of B cell origin. U266 is known to produce monoclonal antibodies and IL-6 (Kawano et al., 1988; Nilsson et al.,). RPMI 8226 produces only immunoglobulin light chains, and there is no evidence for heavy chain or IL-6 production. The MM.1 (also called MM.1S) cell line, established from the peripheral blood cells of a patient with IgA myeloma, secretes lambda light chain, is negative for the presence of EBV genome, and expresses leukocyte antigen DR, PCA-1, T9, and T10 antigens (Goldman-Leikin et al., 1989). MM.1R is a dexamethasone (dex)-resistant variant of MM.1 cells, also known as MM.1S (Moalli et al., 1992), and was kindly provided by Dr. Steven T. Rosen of Northwestern University Medical School (Chicago, Ill.). Human MM cell line OCI was kindly provided by Dr. James Berenson from Cedar-Sinai Hospital (Los Angeles, Calif.).

The rabbit polyclonal antibodies to STAT3, and mouse monoclonal antibodies against phospho-STAT3 were obtained from Santa Cruz Biotechnology (Santa Cruz, Calif.). Goat anti-rabbit-horse radish peroxidase (HRP) conjugate was purchased from Bio-Rad Laboratories (Hercules, Calif.). Guggulsterone with a purity greater than 98%, was purchased from Steraloids. RPMI-1640, fetal bovine serum (FBS), 0.4% trypan blue vital stain, and antibiotic-antimycotic mixture were obtained from Life Technologies, Inc. (Grand Island, N.Y.). Bacteria-derived recombinant human IL-6 was kindly provided by Sandoz Pharmaceutical (East Hanover, N.J.).

Cell culture. All the human multiple myeloma cell lines were cultured in RPMI 1640 medium containing 1X antibiotic-antimycotic. U266, MM.1S, RPMI 8226 and MM.1R were cultured in 10% FBS. Cells were free of mycoplasma contamination as tested by Hoechst staining and by RT-PCR.

Western blot. For detection of STAT proteins, whole-cell extracts were prepared by lysing the guggulsterone-treated cells in lysis buffer (20 mM Tris, pH 7.4, 250 mM NaCl, 2 mM EDTA, pH 8.0, 0.1% Triton −X100, 0.01 mg/ml aprotinin, 0.005 mg/ml leupeptin, 0.4 mM PMSF, and 4 mM NaVO4). Lysates were then spun at 14,000 rpm for 10 min to remove insoluble material, and resolved on a 7.5% gel. After electrophoresis, the proteins were electrotransferred to a nitrocellulose membrane, blocked with 5% nonfat milk, and probed with anti-STAT antibodies (1:1000) overnight at 4° C. The blot was washed, exposed to HRP-conjugated secondary antibodies for 1 h, and finally examined by chemiluminescence (ECL, Amersham Pharmacia Biotech. Arlington Heights, Ill.).

B. Results

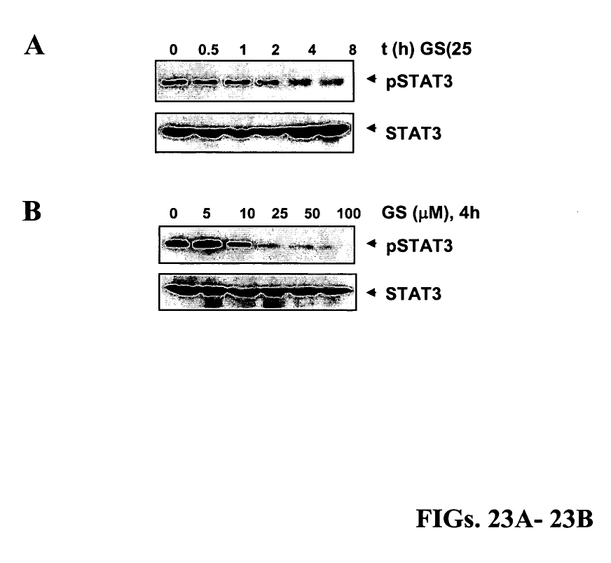

Guggulsterone inhibits constitutive STAT3 phosphorylation in multiple myeloma cells. Guggulsterone was studied to assess inhibition of the constitutive STAT3 phosphorylation in U266. U266 cells were incubated either with different concentrations of guggulsterone for 4 h or with 25 μM guggulsterone for different times. Guggulsterone inhibited the constitutively active STAT3 in a time-(FIG. 23A) and dose-(FIG. 23B) dependent manner. Guggulsterone-induced inhibition could be observed as early as 4 h and with a concentration as low as 25 μM. Guggulsterone treatment did not alter the overall expression of STAT3 protein.

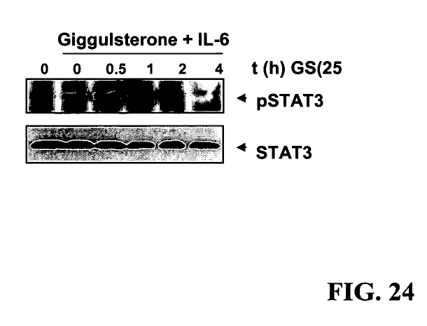

Guggulsterone inhibits IL-6-inducible STAT3 phosphorylation in human multiple myeloma. Since IL-6-induced signals are mediated through STAT3 phosphorylation, the status of STAT3 phosphorylation was examined. All MM cell lines express STAT3 but only U266 expressed a constitutively phosphorylated STAT3. These results are consistent with our previous observations that only U266 constitutively secretes IL-6 (Bharti et al., 2003. Blood 101:1053.). Since IL-6 is a growth factor for MM and induces STAT3 phosphorylation (Kawano et al.,; Klein et al., 1995; Catlett-Falcone et al., 1999), inhibition of IL-6-induced STAT3 phosphorylation by guggulsterone was assessed. RPMI 8226 cells (which do not express constitutively phosphorylated STAT3) were treated with IL-6. IL-6 induced phosphorylation of STAT3 as early as 5 min and began to declined at 60 min (data not shown). RPMI 8226 cells were then incubated with guggulsterone for different times and examined for IL-6-inducible STAT3 phosphorylation. As seen in FIG. 24, IL-6-induced STAT3 phosphorylation was blocked by guggulsterone in a time-dependent manner. Exposure of cells to guggulsterone for 4 h was sufficient to completely suppress IL-6-induced STAT3 phosphorylation. Guggulsterone alone had no effect on STAT3 phosphorylation in these cells (data not shown).

### REFERENCES

The following references were cited herein:
U.S. Pat. No. 4,870,287
U.S. Pat. No. 5,273,747
U.S. Pat. No. 5,466,468
U.S. Pat. No. 5,690,948
U.S. Pat. No. 5,760,395
U.S. Pat. No. 5,972,341
U.S. Pat. No. 6,019,975
U.S. Pat. No. 6,086,889
U.S. Pat. No. 6,113,949
U.S. Pat. No. 6,120,779
U.S. Pat. No. 6,277,396
U.S. Pat. No. 6,436,991
U.S. Pat. No. 6,630,177
U.S. Pat. No. 6,737,442
U.S. Pat. No. 6,896,901
Abu-Amer et al., Nat. Med., 3:1189-1190, 1997.
Aggarwal et al., Indian J. Exp. Biol., 42:341-353, 2004.
Auphan et al., Science, 270(5234):286-90, 1995.
Bharti, et al., Blood, 101(3):1053-1062, 2003.
Blair et al., J. Cell Biol., 102(4):1164-1172, 1986.
Bonizzi, et al., J. Immunol., 159(11):5264-72, 1997.
Brand, et al., J. Clin. Invest., 97(7):1715-1722, 1996.
Bromberg et al., Mol. Cell Biol., 18:2553, 1998.
Bucay et al., Genes Dev., 12(9):1260-1268, 1998.
Catlett-Falcone et al., Immunity, 10: 105, 1999.
Catz, et al., Oncogene, 20:7342-7351, 2001.
Chaturvedi et al., J. Biol. Chem., 269:14575, 1994.
Chaturvedi, et al., Methods Enzymol., 319:585-602
Chu, et al., Proc. Natl. Acad. Sci. USA, 94:10057-10062, 1997.
Craig, et al., Am. J. Clin. Nutr., 70(3 Suppl):491 S-499S, 1999.
Craig, J. Am. Diet. Assoc., 97(10 Suppl 2):S199-204, 1997.
Cui, et al., J. Biol. Chem., 278(12):10214-10220, 2003.
Dai et al., Arthritis, 279:37219, 2004.
Darnell Jr., Science, 277:1630, 1997.
DiDonato et al., Nature, 388:548, 1997.
Dougall et al., Genes Dev., 13(18):2412-2424, 1999.
Emmerich et al., Blood, 94(9):3129-34, 1999.
Esteve et al., J. Biol. Chem., 277:35150-35155, 2002.
Estrov et al., Blood, 94(8):2844-2853, 1999.
Feinman et al., Blood, 93:3044, 1999.
Finco et al., Proc. Natl. Acad. Sci. USA.91 (25):11884-8, 1994.
Forum, Br. J. Haematol., 115:522, 2001.
Franzoso et al., Genes Dev., 11(24):3482-3496, 1997.
Garcia and Jove, J. Biomed. Sci., 5:79, 1998.
Garcia et al., Cell Growth Differ., 8:1267, 1997.
Garg et al., Leukemia, 16(6):1053-1068, 2002.
Giri et al., J. Biol. Chem., 273:14008-14014, 1998.
Goldman-Leikin et al., J Lab. Clin. Med., 113:335, 1989.
Grad et al., Curr. Opin. Oncol., 12:543, 2000.
Grumont et al., Genes Dev., 13:400-411, 1999.
Gujral et al., Indian J. Physiol. Pharmacol., 4:267-273, 1960.
Guttridge et al., Mol. Cell Biol., 19:5785-5799, 1999.
Hallek et al., Blood, 91:3, 1998.
Hsu et al., Cell, 84(2):299-308, 1996.
Hsu et al., Proc. Natl. Acad. Sci. USA, 96:3540-3545, 1999.
Huang et al., Cancer Res., 60(19):5334-9, 2000.
Iotsova et al., Nat. Med., 3(11):1285-1289, 1997.
Johnson et al., Cell, 71(4):577-86, 1992.
Karin et al., Nat. Rev. Cancer, 2(4):301-310, 2002.
Kawano et al., Nature, 332:83, 1988.
Klein et al., Blood, 85:863, 1995.
Kong et al., Nature, 397:315-323, 1999
Kong et al., Nature, 397(6717):315-323, 1999.
Kreuz et al., Mol. Cell Bio., 21:3964-3973, 2001.
Li et al., Trends Biotechnol., 18:151, 2000.
Lomaga et al., Genes Dev., 13(8):1015-1024, 1999.
Mahon et al., J. Biol. Chem., 270(48):28557-64, 1995.
Majumdar and Aggarwal, J. Immunol., 167:2911-2920, 2001.
Manna et al., J. Immunol., 165(9):4927-34, 2000.
Meselhy, Phytochemistry, 62(2):213-218, 2003.
Miagkov et al., Proc. Natl. Acad. Sci. USA, 95:13859-13864, 1998.
Mitchell et al., Ann. NY Acad. Sci., 690:153-166, 1993.
Mitchell et al., J. Clin. Oncol., 8(5):856-869, 1990.
Miyamoto et al., Mol. Cell Biol., 14(5):3276-82, 1994.
Miyamoto et al., Proc. Natl. Acad. Sci. USA, 91 (26):12740-4, 1994.
Mizuno et al., Gene, 215(2):339-343, 1998.
Moalli et al., Blood, 79:213, 1992.
Morton et al., Arch. Surg., 127:392-399, 1992.
Nasuhara et al., J. Biol. Chem., 274(28):19965-72, 1999.
Natarajan and Bright, J. Immunol., 168:6506, 2002.
Newman et al., J. Nat. Prod., 66(7):1022-1037, 2003.
Nilsson et al., Clin. Exp. Immunol., 7:477, 1970.
Pettersson et al., Blood, 79:495, 1992.
Ravindranath and Morton, Intern. Rev. Immunol., 7: 303-329, 1991.
Remington's Pharmaceutical Sciences, 15^{th} ed., pages 1035-1038 and 1570-1580, Mack Publishing Company, Easton, Pa., 1980.
Rosenberg et al., Ann. Surg. 210(4):474-548, 1989.
Rosenberg et al., N. Engl. J. Med., 319:1676, 1988.
Rothwarf and Karin, Science, STKE 1999:RE1, 1999.
Schwenzer et al., J. Biol. Chem., 274:19368-19374, 1999.
Sharma et al., Arzneimittelforschung, 27(7):1455-1457, 1977.
Shevde et al., Proc. Natl. Acad. Sci. USA, 97:7829, 2000.
Shishodia et al., Cancer Res., 63(15):4375-83, 2003.
Shyamala et al., Proc. Natl. Acad. Sci. USA, 89(22):10628-32, 1992.
Simeonidis et al., Proc. Natl. Acad. Sci. USA, 96(1):49-54, 1999.
Sinal and Gonzalez, Trends Endocrinol. Metab., 13:275-276, 2002.
Singh et al., Altern. Ther. Health Med., 9(3):74-79, 2003.
Soriano et al., Cell, 64(4):693-702, 1991.
Stehlik et al., J. Exp. Med., 188:211-216, 1998.
Taga and Kishimoto, Annu. Rev. Immunol., 15:797, 1997.
Takahashi et al., J. Bone Miner Res., 2(4):311-317, 1987.
Takemoto et al., Proc. Natl. Acad. Sci. USA, 94:13897, 1997.
Thurberg et al. Curr. Opin. Lipidol., 9(5):387-396, 1998.
Tu et al., Blood, 88:1805, 1996.
Turkson et al., Mol. Cell Biol., 18:2545, 1998.
Urizar et al., Annu Rev Nutr., 23:303-313, 2003.
Urizar et al., Science, 296(5573): 1703-1706, 2002.
Van Antwerp et al., Science, 274(5288):787-9, 1996.
Wang et al., Science, 274(5288):784-7, 1996.
Weber-Nordt et al., Blood, 88:809, 1996.
Wei et al., Endocrinology, 142:1290, 2001.
Wittekindt et al., Nucleic Acids Res., 28(3):800-8, 2000.
Wu et al., Mol. Endocrinol., 16(7):1590-1597, 2002.
Yamamoto et al., J. Biol. Chem., 270:31315-31320, 1995.
Yamamoto, Curr. Mol. Med., 1(3):287-296, 2001.
Yang and Huang, Gene Therapy, 4 (9):950-960, 1997.
You et al., Mol. Cell Biol ., (17:7328-7341, 1997.
Yuan et al., Science, 293:1673-1677, 2001.
Zhong et al., Mol. Cell, (5):661-71, 1998.
Zhu et al., FEBS Lett., 508:369-374, 2001.
Zong et al., Genes Dev., 13:382-387, 1999.

What is claimed is:

1. A method of inhibiting activation of NF-κB in a cell, comprising the step of contacting the cell with guggulsterone or a guggulsterone analog, wherein guggulsterone or guggulsterone analog suppresses NF-κB activation.

2. The method of claim 1, wherein the NF-κB activation is induced by a carcinogen, an inflammatory stimulus, or both a carcinogen and an inflammatory stimulus.

3. The method of claim 2, wherein the carcinogen is selected from the group consisting of phorbol ester, okadaic acid, and cigarette smoke.

4. The method of claim 2, wherein the inflammatory stimulus is selected from the group consisting of hydrogen peroxide, TNF, and IL-1β.

5. The method of claim 1, wherein the NF-κB activation is constitutive.

6. The method of claim 1, wherein the suppression of NF-κB activation is through inhibition of Akt kinase; inhibition of IκBα kinase; IκBα phosphorylation; IκBα degradation; p65 phosphorylation and nuclear translocation; or combinations thereof.

7. The method of claim 1, wherein the suppression of NF-κB activation by guggulsterone or guggulsterone analog abrogates NF-κB-dependent gene transcription induced by TNF, TNF receptor 1, TNF receptor-associated death domain, TNF receptor-associated factor-2, NF-κB-inducing kinase, IκBα kinase, or any combination thereof.

8. A method of downregulating gene expression in a cell, comprising the step of contacting the cell with guggulsterone or a guggulsterone analog, wherein guggulsterone or guggulsterone analog suppresses NF-κB activation and the suppression of NF-κB activation down-regulates the expression of anti-apoptotic genes, proliferative genes, metastasis genes, or combinations thereof.

9. The method of claim 8, wherein the anti-apoptotic gene is selected from the group consisting of IAP1, XIAP, Bfl-1/A1, bcl-2, cFLIP and survivin.

10. The method of claim 8, wherein the proliferative gene is cyclin D1, c-myc, or cyclin D1 and c-myc.

11. The method of claim 8, wherein the metastasis gene is selected from the group consisting of MMP-9, COX-2 and VEGF.

12. A method of enhancing the effect of an apoptosis-inducing agent in a cell, comprising the step of contacting the cell with the apoptosis-inducing agent, and guggulsterone or a guggulsterone analog, wherein inhibition of NF-κB activation by guggulsterone results in enhanced apoptosis.

13. The method of claim 12, wherein the apoptosis-inducing agent is TNF, a cancer therapeutic, a chemotherapeutic agent, or a combination thereof.

14. The method of claim 13, wherein the chemotherapeutic agent is selected from the group consisting of paclitaxel, doxorubicin, gemcitabine, 5-Flurouracil, etoposide, cisplatin, campothecin, and vincristine.

15. A method of treating a cancerous or pre-cancerous state in an individual in need of such treatment, comprising the step of administering a pharmacologically effective dose of guggulsterone to the individual.

16. The method of claim 15, further comprising the step of administering a non-guggulsterone inhibitor of the activation of NF-κB, wherein the non-guggulsterone inhibitor is selected from the group consisting of curcumin, CAPE, capsaicin, sanguinarin, PTPase inhibitors, lapachone, resveratrol, vesnarinone, leflunomide, anethole, PI3 kinase inhibitors, oleanderin, emodin, serine protease inhibitors, protein tyrosine kinase inhibitors, thalidomide and methotrexate.

18. The method of claim 15, wherein the cancerous or pre-cancerous state is characterized by at least one cell having constitutive activation of NF-κB.

19. The method of claim 15, wherein the cancerous or pre-cancerous state is selected from the group consisting of breast cancer, prostate cancer, melanoma, pancreatic cancer, colon cancer, leukemia and multiple myeloma.

20. A method of increasing cytotoxic effects of a chemotherapeutic agent against cancer in an individual, comprising the step of administering to the individual the chemotherapeutic agent and an inhibitor of the activation of NF-κB, wherein the inhibitor of the activation of NF-κB increases the cytotoxic effects of the chemotherapeutic agent against cancer cells in the individual.

21. The method of claim 20, wherein the inhibitor of the activation of NF-κB is selected from the group consisting of guggulsterone, guggulsterone analog, curcumin, CAPE, capsaicin, sanguinarin, PTPase inhibitors, lapachone, resveratrol, vesnarinone, leflunomide, anethole, PI3 kinase inhibitors, oleanderin, emodin, serine protease inhibitors, protein tyrosine kinase inhibitors, thalidomide and methotrexate.

23. The method of claim 20, wherein the chemotherapeutic agent is selected from the group consisting of paclitaxel, doxorubicin, gemcitabine, 5-Flurouracil, etoposide, cisplatin, campothecin, and vincristine.

24. The method of claim 20, wherein the cancer is selected from the group consisting of breast cancer, prostate cancer, melanoma, pancreatic cancer, colon cancer, leukemia and multiple myeloma.

25. A method of inhibiting osteoclastogenesis comprising the step of contacting the cell with guggulsterone or a guggulsterone analog, wherein guggulsterone or guggulsterone analog suppresses NF-κB activation and osteoclastogenesis.

26. The method of claim 25, wherein the NF-κB activation and osteoclastogenesis is induced by RANKL.


SEQUENCE LISTING (6 sequences)

SEQ ID NO: 1 - DNA, 45 bp, Artificial Sequence
  Description of Artificial Sequence  Synthetic Primer
ttgttacaagggactttccgctggggactttccagggaggcgtgg

SEQ ID NO: 2 - DNA, 45 bp, Artificial Sequence
  Description of Artificial Sequence  Synthetic Primer
ttgttacaactcactttccgctgctcactttccagggaggcgtgg

SEQ ID NO: 3 - DNA, 19 bp, Artificial Sequence
  Description of Artificial Sequence  Synthetic Primer
gagtctcttatttattttt

SEQ ID NO: 4 - DNA, 25 bp, Artificial Sequence
  Description of Artificial Sequence  Synthetic Primer
gctgctgaggagttcctggacgtgc

SEQ ID NO: 5 - DNA, 45 bp, Artificial Sequence; identical to SEQ ID NO: 1
  Description of Artificial Sequence  Synthetic Primer

SEQ ID NO: 6 - DNA, 45 bp, Artificial Sequence; identical to SEQ ID NO: 2
  Description of Artificial Sequence  Synthetic Primer